(19) United States
(12) Patent Application Publication
(10) Pub. No.: US 2024/0268649 A1
(43) Pub. Date: Aug. 15, 2024

(54) INTEGRATED CABLE CONNECTOR FOR LUMINOUS EFFICIENCY

(71) Applicant: Cilag GmbH International, Zug (CH)

(72) Inventors: Jeremiah D. Henley, Fair Oaks, CA (US); Dillon Karg, Santa Clara, CA (US); Preston Manwaring, Farmington, UT (US)

(73) Assignee: Cilag GmbH International, Zug (CH)

(21) Appl. No.: 18/167,020

(22) Filed: Feb. 9, 2023

(51) Int. Cl. A61B 1/06; A61B 1/00; A61B 1/07

(52) U.S. Cl. CPC A61B 1/0638; A61B 1/00117; A61B 1/00126; A61B 1/0684; A61B 1/07; G02B 6/403

(57) ABSTRACT

Advanced systems for endoscopic visualization with illumination for color visualization and advanced visualization. A device for connecting optical and electrical components of an imaging system includes a first fiber optic ferrule dedicated to receiving a first emission of electromagnetic radiation emitted by a first source, and a second fiber optic ferrule dedicated to receiving a second emission of electromagnetic radiation emitted by a second source. The device includes a data connection component configured to receive bidirectional data communications. The imaging system is such that the first source is tuned to pulse a different waveband of electromagnetic radiation than the second source.

### TECHNICAL FIELD

This disclosure is directed to advanced visualization and digital imaging systems and methods and, more particularly but not entirely, to means for increasing and maintaining luminous efficiency when transmitting different types of electromagnetic radiation.

### BACKGROUND

Endoscopic surgical instruments are often preferred over traditional open surgical devices because the small incision tends to reduce post-operative recovery time and associated complications. In some instances of endoscopic visualization, it is desirable to view a space with high-definition color imaging and further with one or more advanced visualization techniques providing additional information that cannot be discerned with the human eye. However, the space constrained environment of an endoscope introduces numerous technical challenges when seeking to capture advanced visualization data in a light deficient environment of a surgical scene.

There are numerous endoscopic visualization systems seeking to capture advanced visualization data, such as multispectral data, fluorescence data, and mapping data, while working within the space constrained environment of an endoscope. However, these systems do not address the technical challenges associated with transmitting different types of electromagnetic radiation, which may include white light and narrowband electromagnetic radiation selected for multispectral or fluorescence visualization. Specifically, these systems do not describe means for increasing and maintaining luminous efficiency when transmitting the different types of electromagnetic radiation.

For example, commonly owned U.S. Patent Application Publication No. 2020/0404131, entitled “HYPERSPECTRAL AND FLUORESCENCE IMAGING WITH TOPOLOGY LASER SCANNING IN A LIGHT DEFICIENT ENVIRONMENT,” filed on Oct. 24, 2019, which is incorporated by reference in its entirety, describes an endoscopic visualization system for color and “specialty” imaging. In this disclosure, an emitter is configured to emit electromagnetic energy in wavelength bands within the visible spectrum, including red, green, and blue emissions, as well as specialty emissions, wherein the specialty emissions may include hyperspectral, fluorescence, or laser mapping emissions of electromagnetic energy. However, this disclosure does not address illumination losses and inefficiencies when emitting the electromagnetic radiation within the visible spectrum, or the specialty emissions for hyperspectral, fluorescence, and laser mapping imaging.

Additionally, U.S. Pat. No. 10,631,713, entitled “MULTI-STAGE INSTRUMENT CONNECTOR,” filed on Mar. 17, 2015, which is incorporated by reference in its entirety, describes a medical device that connectors for coupling surgical instruments to input/output sources. This disclosure indicates that the connector may include a first pathway for transmitting high intensity illumination, and a second pathway for transmitting imaging controls and captured image data. However, this disclosure does not describe an integrated connector that includes two or more dedicated channels for transmitting different types of electromagnetic radiation, and an additional channel for transmitting data and control signals.

In view of the foregoing, disclosed herein are systems, methods, and devices for improving efficiency of illumination pathways within an endoscopic visualization system capable of color and advanced visualization.

### BRIEF DESCRIPTIONS OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a schematic illustration of an example system for endoscopic visualization with color imaging and advanced imaging;

FIG. 1B is a schematic illustration of an example image pickup portion of a system for endoscopic visualization with color imaging and advanced imaging

FIG. 1C is a schematic illustration of an example emitter and controller of a system for endoscopic visualization with color imaging and advanced imaging;

FIG. 2A is a schematic block diagram of an example data flow for a time-sequenced visualization system;

FIG. 2B is a schematic block diagram of an example data flow for a time-sequenced visualization system;

FIG. 2C is a schematic flow chart diagram of a data flow for capturing and reading out data for a time-sequenced visualization system;

FIG. 3A is a schematic block diagram of an example system for processing data output by an image sensor with a controller in communication with an emitter and the image sensor;

FIG. 3B is a schematic block diagram of an example system for processing data output by an image sensor to generate color imaging data and advanced imaging data;

FIG. 3C is a schematic block diagram of an example system for processing data through a memory buffer to provide data frames to an image signal processor at regular intervals;

FIG. 4 is a schematic diagram of an illumination system for illuminating a light deficient environment according to a variable pulse cycle;

FIG. 5A is a schematic illustration of an illumination and visualization controller for endoscopic visualization system;

FIG. 5B is a schematic illustration of an illumination and visualization controller for endoscopic visualization system;

FIG. 5C is a schematic illustration of an illumination and visualization controller for endoscopic visualization system;

FIG. 6 is a perspective view of an example integrated cable connector that separates transmissions of visible electromagnetic radiation and narrowband electromagnetic radiation to increase luminous efficiency;

FIG. 7 is a schematic illustration of an integrated cable connector interfacing with an illumination and visualization controller;

FIG. 8A is a schematic illustration of a cross-sectional view of a transverse section of a lumen of an endoscope, with optical fibers distributed around visualization components;

FIG. 8B is a schematic illustration of a cross-sectional view of a transverse section of a lumen of an endoscope, with optical fibers distributed around visualization components;

FIGS. 9A-9D illustrate straight-on views of example implementations of an integrated cable connector;

FIG. 10A is a schematic illustration of an example mapping pattern comprising a grid array;

FIG. 10B is a schematic illustration of an example mapping pattern comprising a dot array;

FIG. 11 illustrates a portion of the electromagnetic spectrum divided into a plurality of different wavebands which may be pulsed by sources of electromagnetic radiation of an emitter,

FIG. 12 is a schematic diagram illustrating a timing sequence for emission and readout for generating data frames in response to pulses of electromagnetic radiation; and

FIG. 13 is a schematic block diagram of an example computing device.

### DETAILED DESCRIPTION

Disclosed herein are systems, methods, and devices for digital visualization that may be primarily suited to medical applications such as medical endoscopic imaging. An embodiment of the disclosure is an endoscopic system for color visualization and “advanced visualization” of a scene. The advanced visualization includes one or more of multispectral imaging, fluorescence imaging, or topographical mapping. Data retrieved from the advanced visualization may be processed by one or more algorithms configured to determine characteristics of the scene. The advanced visualization data may specifically be used to identify tissue structures within a scene, generate a three-dimensional topographical map of the scene, calculate dimensions of objects within the scene, identify margins and boundaries of different tissue types, and so forth.

Specifically disclosed herein are systems, methods, and devices for increasing luminous efficiency when transmitting different types of electromagnetic radiation (“EMR”), including visible EMR and narrowband EMR selected for multispectral or fluorescence visualization. An integrated cable described herein includes two or more independent fiber optic ferrules that are each dedicated to receiving and transmitting a certain type of EMR. In an implementation, a first fiber optic ferrule is dedicated to receiving only visible EMR, and a second fiber optic ferrule is dedicated to receiving only narrowband EMR selected for fluorescence or multispectral visualization. Each of the fiber optic ferrules is in communication with a plurality of optical fibers configured to transmit the respective EMR to a distal end of an endoscope to illuminate a scene.

An embodiment of the disclosure is an endoscopic visualization system that includes an emitter, an image sensor, and a controller. The emitter includes a plurality of separate and independently actuatable sources of EMR that may be separately cycled on and off to illuminate a scene with pulses of EMR. The image sensor accumulates EMR and reads out data for generating a plurality of data frames. The controller synchronizes operations of the emitter and the image sensor to output a desired visualization scheme based on user input. The visualization scheme may include a selection of one or more of color imaging, multispectral imaging, fluorescence imaging, topographical mapping, or anatomical measurement.

The controller instructs the emitter and the image sensor to operate in a synchronized sequence to output a video stream that includes one or more types of visualization (i.e., color imaging, multispectral imaging, fluorescence imaging, topographical mapping, or anatomical measurement). The controller instructs the emitter to actuate one or more of the plurality of EMR sources to pulse according to a variable pulse cycle. The controller instructs the image sensor to accumulate EMR and read out data according to a variable sensor cycle that is synchronized in time with the variable pulse cycle. The synchronized sequence of the emitter and the image sensor enables the image sensor to read out data corresponding with a plurality of different visualization types. For example, the image sensor may read out a color frame in response to the emitter pulsing a white light or other visible EMR, the image sensor may readout a multispectral frame in response to the emitter pulsing a multispectral waveband of EMR, the image sensor may read out data for calculating a three-dimensional topographical map in response to the emitter pulsing EMR in a mapping pattern, and so forth.

The controller optimizes and adjusts a sensor cycle of an image sensor to output data frames for color imaging and/or advanced imaging at a sufficient rate, while ensuring the pixel array accumulates a sufficient amount of EMR for each data frame. The controller may instruct the image sensor to implement pixel binning on a per-frame basis, such that the image sensor implements pixel binning for some data frames and reads out all pixels for other data frames. In some cases, the controller instructs the image sensor to read out all pixels and thereby output a high-definition color data frame in response to the emitter pulsing white EMR. The controller may further instruct the image sensor to bin the pixel array and read out fewer pixels in response to the emitter pulsing EMR for advanced visualization, such as multispectral imaging, fluorescence imaging, or topographical mapping.

The controller may additionally optimize and adjust the variable pulse cycle in real-time based on user input, sufficient exposure of resultant data frames, and inherent properties of a corresponding pixel array. In some cases, a pixel array has varying sensitivities to different wavebands of EMR. In these cases, the pixel array is irradiated with EMR for shorter or longer durations of time depending on the type of illumination pulse to ensure the pixel array outputs data frames with consistent exposure levels. The controller adjusts the irradiation time of the pixel array and the pulsing duration of the emitter in real-time to compensate for the pixel array's varying efficiencies to different types of illumination.

The systems, methods, and devices described herein are implemented for color visualization and advanced visualization. The advanced visualization techniques described herein can be used to identify certain tissues, see through tissues in the foreground, calculate a three-dimensional topography of a scene, and calculate dimensions and distances for objects within the scene. The advanced visualization techniques described herein specifically include multispectral visualization, fluorescence visualization, and laser mapping visualization.

### Multispectral Visualization

Spectral imaging uses multiple bands across the electromagnetic spectrum. This is different from conventional cameras that only capture light across the three wavelengths based in the visible spectrum that are discernable by the human eye, including the red, green, and blue wavelengths to generate an RGB image. Spectral imaging may use any wavelength bands in the electromagnetic spectrum, including infrared wavelengths, the visible spectrum, the ultraviolet spectrum, x-ray wavelengths, or any suitable combination of various wavelength bands. Spectral imaging may overlay imaging generated based on non-visible bands (e.g., infrared) on top of imaging based on visible bands (e.g., a standard RGB image) to provide additional information that is easily discernable by a person or computer algorithm.

The multispectral imaging techniques discussed herein can be used to “see through” layers of tissue in the foreground of a scene to identify specific types of tissue and/or specific biological or chemical processes. Multispectral imaging can be used in the medical context to quantitatively track the process of a disease and to determine tissue pathology. Additionally, multispectral imaging can be used to identify critical structures such as nerve tissue, muscle tissue, cancerous cells, blood vessels, and so forth. In an embodiment, multispectral partitions of EMR are pulsed and data is gathered regarding the spectral responses of different types of tissue in response to the partitions of EMR. A datastore of spectral responses can be generated and analyzed to assess a scene and predict which tissues are present within the scene based on the sensed spectral responses.

Multispectral imaging enables numerous advantages over conventional imaging. The information obtained by multispectral imaging enables medical practitioners and/or computer-implemented programs to precisely identify certain tissues or conditions that may not be possible to identify with RGB imaging. Additionally, multispectral imaging may be used during medical procedures to provide image-guided surgery that enables a medical practitioner to, for example, view tissues located behind certain tissues or fluids, identify atypical cancerous cells in contrast with typical healthy cells, identify certain tissues or conditions, identify critical structures, and so forth. Multispectral imaging provides specialized diagnostic information about tissue physiology, morphology, and composition that cannot be generated with conventional imaging.

### Fluorescence Visualization

Fluorescence occurs when an orbital electron of a molecule, atom, or nanostructure is excited by light or other EMR, and then relaxes to its ground state by emitting a photon from the excited state. The specific frequencies of EMR that excite the orbital electron, or are emitted by the photon during relaxation, are dependent on the particular atom, molecule, or nanostructure. In most cases, the light emitted by the substance has a longer wavelength, and therefore lower energy, than the radiation that was absorbed by the substance.

Fluorescence imaging is particularly useful in biochemistry and medicine as a non-destructive means for tracking or analyzing biological molecules. The biological molecules, including certain tissues or structures, are tracked by analyzing the fluorescent emission of the biological molecules after being excited by a certain wavelength of EMR. However, relatively few cellular components are naturally fluorescent. In certain implementations, it may be desirable to visualize a certain tissue, structure, chemical process, or biological process that is not intrinsically fluorescent. In such an implementation, the body may be administered a dye or reagent that may include a molecule, protein, or quantum dot having fluorescent properties. The reagent or dye may then fluoresce after being excited by a certain wavelength of EMR. Different reagents or dyes may include different molecules, proteins, and/or quantum dots that will fluoresce at particular wavelengths of EMR. Thus, it may be necessary to excite the reagent or dye with a specialized band of EMR to achieve fluorescence and identify the desired tissue, structure, or process in the body.

The fluorescence imaging techniques described herein may be used to identify certain materials, tissues, components, or processes within a body cavity or other light deficient environment. Fluorescence imaging data may be provided to a medical practitioner or computer-implemented algorithm to enable the identification of certain structures or tissues within a body. Such fluorescence imaging data may be overlaid on black-and-white or RGB images to provide additional information and context.

The fluorescence imaging techniques described herein may be implemented in coordination with fluorescent reagents or dyes. Some reagents or dyes are known to attach to certain types of tissues and fluoresce at specific wavelengths of the electromagnetic spectrum. In an implementation, a reagent or dye is administered to a patient that is configured to fluoresce when activated by certain wavelengths of light. The visualization system disclosed herein is used to excite and fluoresce the reagent or dye. The fluorescence of the reagent or dye is detected by an image sensor to aid in the identification of tissues or structures in the body cavity. In an implementation, a patient is administered a plurality of reagents or dyes that are each configured to fluoresce at different wavelengths and/or provide an indication of different structures, tissues, chemical reactions, biological processes, and so forth. In such an implementation, the visualization system described herein emits each of the applicable wavelengths to fluoresce each of the applicable reagents or dyes. This may negate the need to perform individual imaging procedures for each of the plurality of reagents or dyes.

### Laser Mapping Visualization

Laser mapping generally includes the controlled deflection of laser beams. Laser mapping can be implemented to generate one or more of a three-dimensional topographical map of a scene, calculate distances between objects within the scene, calculate dimensions of objects within the scene, track the relative locations of tools within the scene, and so forth.

Laser mapping combines controlled steering of laser beams with a laser rangefinder. By taking a distance measurement at every direction, the laser rangefinder can rapidly capture the surface shape of objects, tools, and landscapes. Construction of a full three-dimensional topography may include combining multiple surface models that are obtained from different viewing angles. Various measurement systems and methods exist in the art for applications in archaeology, geography, atmospheric physics, autonomous vehicles, and others. One such system includes light detection and ranging (LIDAR), which is a three-dimensional mapping system. LIDAR has been applied in navigation systems such as airplanes or satellites to determine position and orientation of a sensor in combination with other systems and sensors. LIDAR uses active sensors to illuminate an object and detect energy that is reflected off the object and back to a sensor.

As discussed herein, the term “laser mapping” includes laser tracking. Laser tracking, or the use of lasers for tool tracking, measures objects by determining the positions of optical targets held against those objects. Laser trackers can be accurate to the order of 0.025 mm over a distance of several meters. The visualization system described herein pulses EMR for use in conjunction with a laser tracking system such that the position of tools within a scene can be tracked and measured.

The endoscopic visualization system described herein implements laser mapping imaging to determine precise measurements and topographical outlines of a scene. In one implementation, mapping data is used to determine precise measurements between, for example, structures or organs in a body cavity, devices, or tools in the body cavity, and/or critical structures in the body cavity. As discussed herein, the term “mapping” encompasses technologies referred to as laser mapping, laser scanning, topographical scanning, three-dimensional scanning, laser tracking, tool tracking, and others. A mapping data frame as discussed herein includes data for calculating one or more of a topographical map of a scene, dimensions of objects or structures within a scene, distances between objects or structures within the scene, relative locations of tools or other objects within the scene, and so forth.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

As used herein, the term “proximal” shall refer broadly to the concept of a portion nearest an origin.

As used herein, the term “distal” shall generally refer to the opposite of proximal, and thus to the concept of a portion farther from an origin, or a farthest portion, depending upon the context.

As used herein, color sensors are sensors known to have a color filter array (CFA) thereon to filter the incoming EMR into its separate components. In the visual range of the electromagnetic spectrum, such a CFA may be built on a Bayer pattern or modification thereon to separate green, red, and blue spectrum components of visible EMR.

As used herein, a monochromatic sensor refers to an unfiltered imaging sensor comprising color-agnostic pixels.

The systems, methods, and devices described herein are specifically optimized to account for variations between “stronger” electromagnetic radiation (EMR) sources and “weaker” EMR sources. In some cases, the stronger EMR sources are considered “stronger” based on the inherent qualities of a pixel array, e.g., if a pixel array is inherently more sensitive to detecting EMR emitted by the stronger EMR source, then the stronger EMR source may be classified as “stronger” when compared with another EMR source. Conversely, if the pixel array is inherently less sensitive to detecting EMR emitted by the weaker EMR source, then the weaker EMR source may be classified as “weaker” when compared with another EMR source. Additionally, a “stronger” EMR source may have a higher amplitude, greater brightness, or higher energy output when compared with a “weaker” EMR source. The present disclosure addresses the disparity between stronger EMR sources and weaker EMR sources by adjusting a pulse cycle of an emitter to ensure a pixel array has sufficient time to accumulate a sufficient amount of EMR corresponding with each of a stronger EMR source and a weaker EMR source.

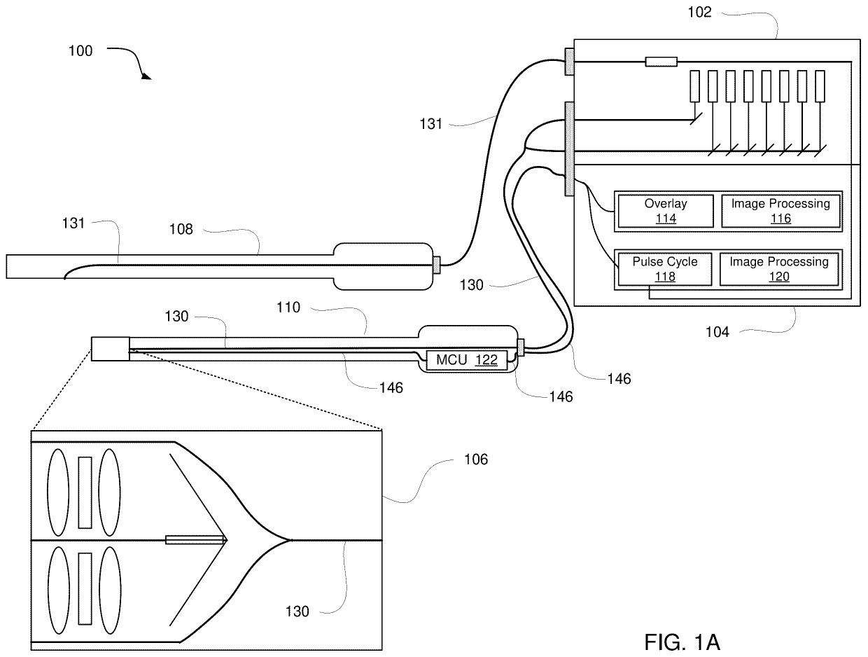

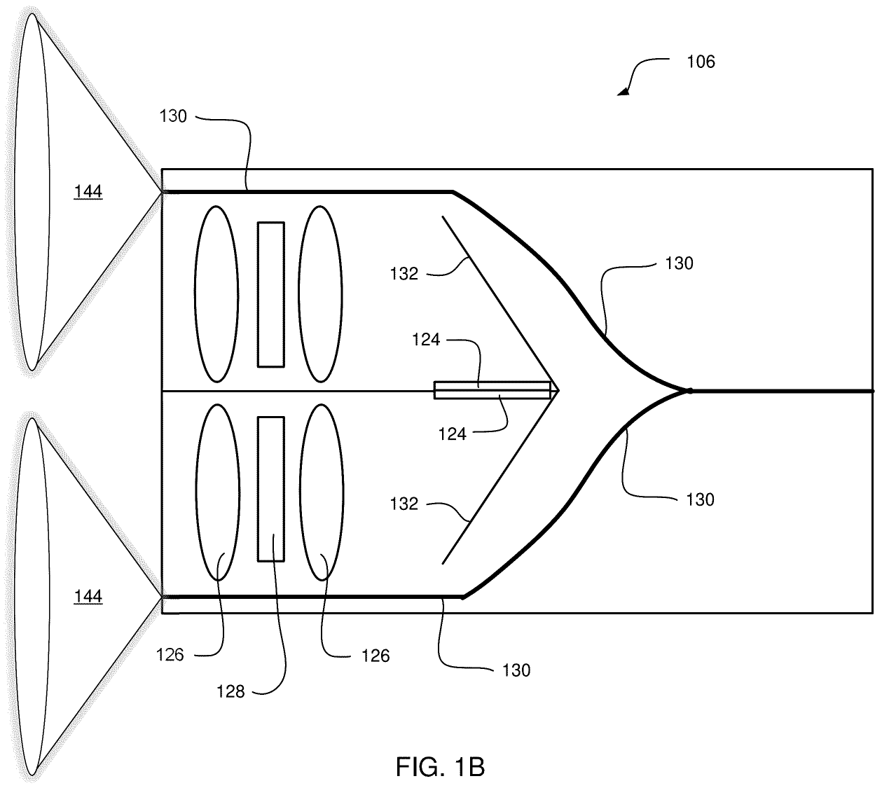

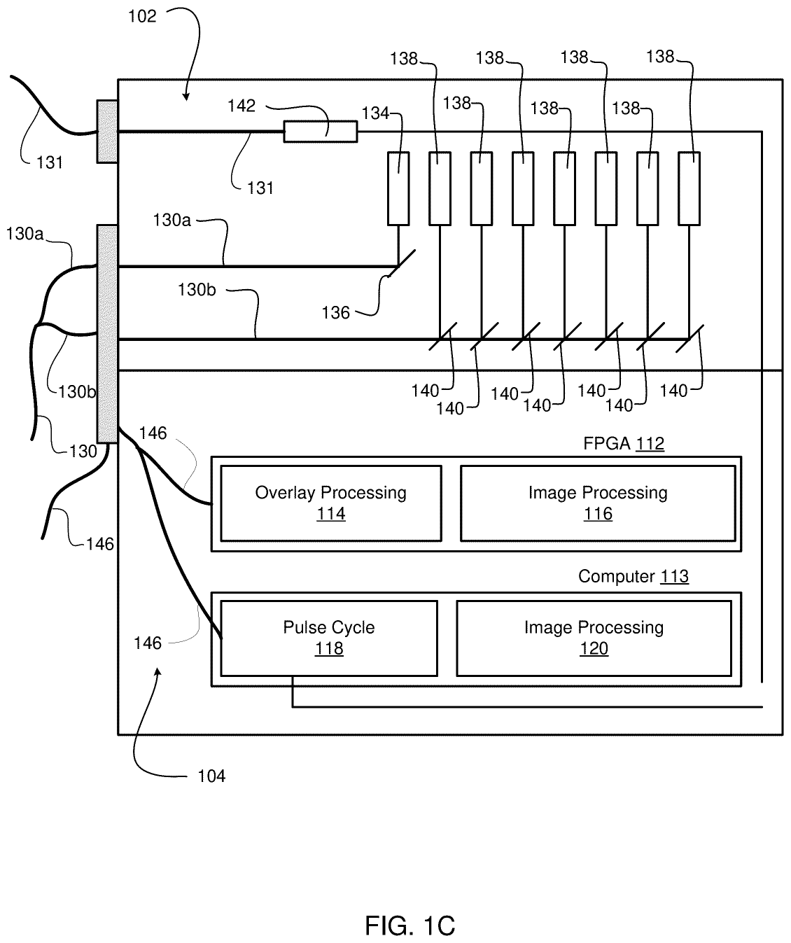

Referring now to the figures, FIGS. 1A-1C illustrate schematic diagrams of a system 100 for endoscopic visualization. The system 100 includes an emitter 102, a controller 104, and an optical visualization system 106. The system 100 includes one or more tools 108, which may include endoscopic tools such as forceps, brushes, scissors, cutters, burs, staplers, ligation devices, tissue staplers, suturing systems, and so forth. The system 100 includes one or more endoscopes 110 such as arthroscopes, bronchoscopes, colonoscopes, colposcopes, cystoscopes, esophagoscope, gastroscopes, laparoscopes, laryngoscopes, neuroendoscopes, proctoscopes, sigmoidoscopes, thoracoscopes, and so forth.

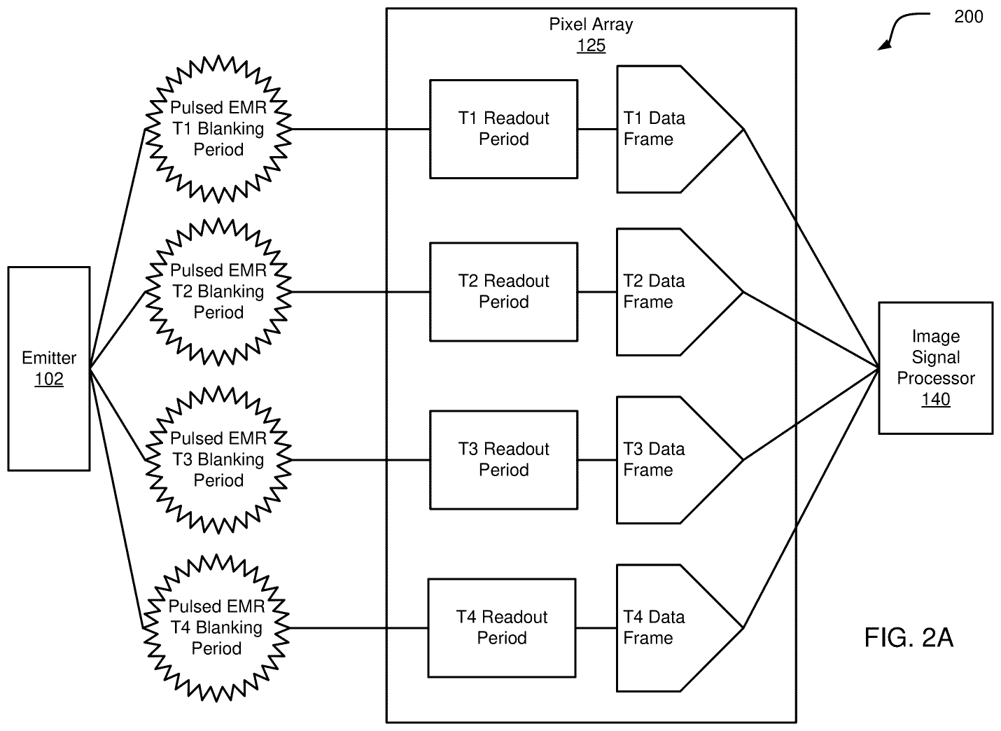

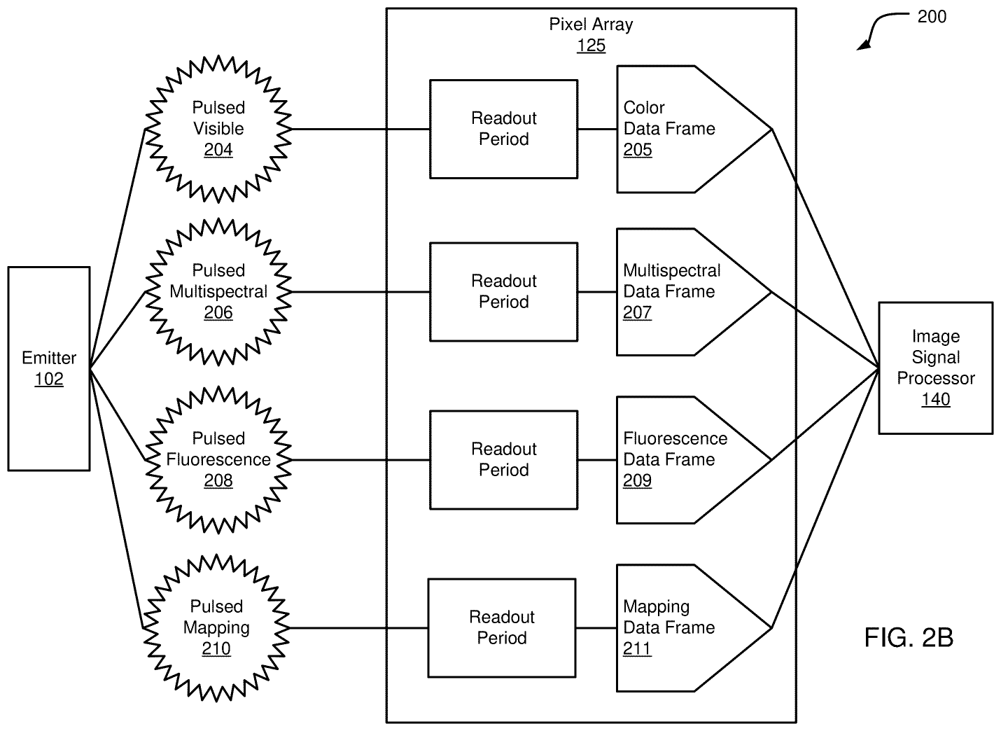

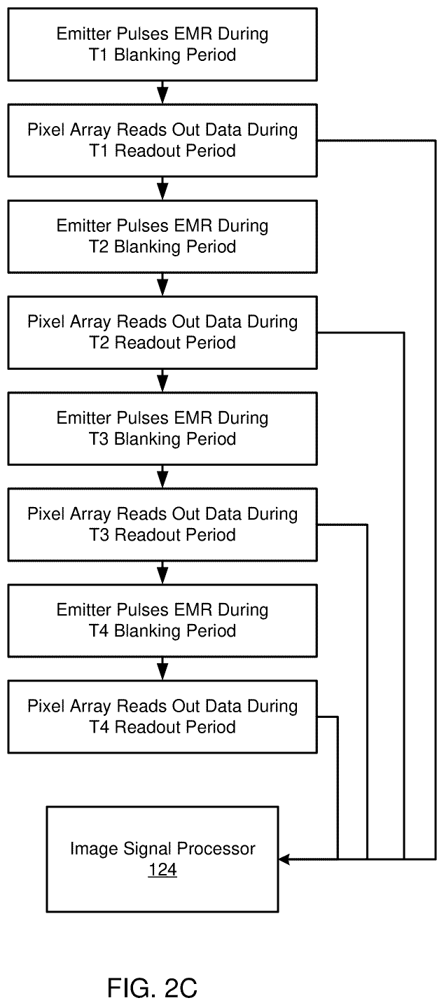

The optical visualization system 106 may be disposed at a distal end of a lumen of an endoscope 110. Alternatively, one or more components of the optical visualization system 106 may be disposed at a proximal end of the lumen of the endoscope 110 or in another region of the endoscope 110. The optical visualization system 106 may include one or more image sensors 124 that each include a pixel array (see pixel array 125 first illustrated in FIG. 2A). The optical visualization system 106 may include one or more lenses 126 and filters 128 and may further include one or more prisms 132 for reflecting EMR on to the pixel array 125 of the one or more image sensors 124. The system 100 may include a waveguide 130 configured to transmit EMR from the emitter 102 to a distal end of the endoscope 110 to illuminate a light deficient environment for visualization, such as within a surgical scene. The system 100 may further include a waveguide 131 configured to transmit EMR from the emitter 102 to a termination point on the tool 108, which may specifically be actuated for laser mapping imaging and tool tracking as described herein.

The optical visualization system 106 may specifically include two lenses 126 dedicated to each image sensor 124 to focus EMR on to a rotated image sensor 124 and enable a depth view. The filter 128 may include a notch filter configured to block unwanted reflected EMR. In a particular use-case, the unwanted reflected EMR may include a fluorescence excitation wavelength that was pulsed by the emitter 102, wherein the system 100 wishes to only detect a fluorescence relaxation wavelength emitted by a fluorescent reagent or tissue.

The image sensor 124 includes one or more image sensors, and the example implementation illustrated in FIGS. 1A-1B illustrates an optical visualization system 106 comprising two image sensors 124. The image sensor 124 may include a CMOS image sensor and may specifically include a high-resolution image sensor configured to read out data according to a rolling readout scheme. The image sensors 124 may include a plurality of different image sensors that are tuned to collect different wavebands of EMR with varying efficiencies. In an implementations, the image sensors 124 include separate image sensors that are optimized for color imaging, fluorescence imaging, multispectral imaging, and/or topographical mapping.

The emitter 102 includes one or more EMR sources, which may include, for example, lasers, laser bundles, light emitting diodes (LEDs), electric discharge sources, incandescence sources, electroluminescence sources, and so forth. In some implementations, the emitter 102 includes at least one white EMR source 134 (may be referred to herein as a white light source). The emitter 102 may additionally include one or more EMR sources 138 that are tuned to emit a certain waveband of EMR. The EMR sources 138 may specifically be tuned to emit a waveband of EMR that is selected for multispectral or fluorescence visualization. The emitter 102 may additionally include one or more mapping sources 142 that are configured to emit EMR in a mapping pattern such as a grid array or dot array selected for capturing data for topographical mapping or anatomical measurement.

The one or more white EMR sources 134 emit EMR into a dichroic mirror 136 that feeds the white EMR into a waveguide 130, which may specifically include a fiber optic cable or other means for carrying EMR to the endoscope. The white EMR source 134 may specifically feed into a first waveguide 130a dedicated to white EMR. The EMR sources 138 emit EMR into independent dichroic mirrors 140 that each feed EMR into the waveguide 130 and may specifically feed into a second waveguide 130b. The first waveguide 130a and the second waveguide 130b later merge into a waveguide 130 that transmits EMR to a distal end of the endoscope 110 to illuminate a scene with an emission of EMR 144.

The one or more EMR sources 138 that are tuned to emit a waveband of EMR may specifically be tuned to emit EMR that is selected for multispectral or fluorescence visualization. In some cases, the EMR sources 138 are finely tuned to emit a central wavelength of EMR with a tolerance threshold not exceeding ±5 nm, ±4 nm, ±3 nm, ±2 nm, or ±1 nm. The EMR sources 138 may include lasers or laser bundles that are separately cycled on and off by the emitter 102 to pulse the emission of EMR 144 and illuminate a scene with a finely tuned waveband of EMR.

The one or more mapping sources 142 are configured to pulse EMR in a mapping pattern, which may include a dot array, grid array, vertical hashing, horizontal hashing, pin grid array, and so forth. The mapping pattern is selected for laser mapping imaging to determine one or more of a three-dimensional topographical map of a scene, a distance between two or more objects within a scene, a dimension of an object within a scene, a location of a tool 108 within the scene, and so forth. The EMR pulsed by the mapping source 142 is diffracted to spread the energy waves according to the desired mapping pattern. The mapping source 142 may specifically include a device that splits the EMR beam with quantum-dot-array diffraction grafting. The mapping source 142 may be configured to emit low mode laser light.

The controller 104 (may be referred to herein as a camera control unit or CCU) may include a field programmable gate array (FGPA) 112 and a computer 113. The FGPA 112 may be configured to perform overlay processing 114 and image processing 116. The computer 113 may be configured to generate a pulse cycle 118 for the emitter 102 and to perform further image processing 120. The FGPA 112 receives data from the image sensor 124 and may combine data from two or more data frames by way of overlay processing 114 to output an overlay image frame. The computer 113 may provide data to the emitter 102 and the image sensor 124. Specifically, the computer 113 may calculate and adjust a variable pulse cycle to be emitted by the emitter 102 in real-time based on user input. Additionally, the computer 113 may receive data frames from the image sensor 124 and perform further image processing 120 on those data frames.

The controller 104 may be in communication with a network, such as the Internet, and automatically upload data to the network for remote storage. The MCU 122 and image sensors 124 maybe exchanged and updated and continue to communicate with an established controller 104. In some cases, the controller 104 is “out of date” with respect to the MCU 122 but will still successfully communicate with the MCU 122. This may increase the data security for a hospital or other healthcare facility because the existing controller 104 may be configured to undergo extensive security protocols to protect patient data.

The controller 104 may communicate with a microcontroller unit (MCU) 122 disposed within a handpiece of the endoscope and/or the image sensor 124 by way of a data transmission pipeline 146. The data transmission pipeline 146 may include a data connection port disposed within a housing of the emitter 102 or the controller 104 that enables a corresponding data cable to carry data to the endoscope 110. In another embodiment, the controller 104 wirelessly communicates with the MCU 122 and/or the image sensor 124 to provide instructions for upcoming data frames. One frame period includes a blanking period and a readout period. Generally speaking, the pixel array 125 accumulates EMR during the blanking period and reads out pixel data during the readout period. It will be understood that a blanking period corresponds to a time between a readout of a last row of active pixels in the pixel array of the image sensor and a beginning of a next subsequent readout of active pixels in the pixel array. Additionally, the readout period corresponds to a duration of time when active pixels in the pixel array are being read. Further, the controller 104 may write correct registers to the image sensor 124 to adjust the duration of one or more of the blanking period or the readout period for each frame period on a frame-by-frame basis within the sensor cycle as needed.

The controller 104 may reprogram the image sensor 124 for each data frame to set a required blanking period duration and/or readout period duration for a subsequent frame period. In some cases, the controller 104 reprograms the image sensor 124 by first sending information to the MCU 122, and then the MCU 122 communicates directly with the image sensor 124 to rewrite registers on the image sensor 124 for an upcoming data frame.

The MCU 122 may be disposed within a handpiece portion of the endoscope 110 and communicate with electronic circuitry (such as the image sensor 124) disposed within a distal end of a lumen of the endoscope 110. The MCU 122 receives instructions from the controller 104, including an indication of the pulse cycle 118 provided to the emitter 102 and the corresponding sensor cycle timing for the image sensor 124. The MCU 122 executes a common Application Program Interface (API). The controller 104 communicates with the MCU 122, and the MCU 122 executes a translation function that translates instructions received from the controller 104 into the correct format for each type of image sensor 124. In some cases, the system 100 may include multiple different image sensors that each operate according to a different “language” or formatting, and the MCU 122 is configured to translate instructions from the controller 104 into each of the appropriate data formatting languages. The common API on the MCU 122 passes information by the scene, including, for example parameters pertaining to gain, exposure, white balance, setpoint, and so forth. The MCU 122 runs a feedback algorithm to the controller 104 for any number of parameters depending on the type of visualization.

The MCU 122 stores operational data and images captured by the image sensors 124.

In some cases, the MCU 122 does not need to continuously push data up the data chain to the controller 104. The data may be set once on the microcontroller 122, and then only critical information may be pushed through a feedback loop to the controller 104. The MCU 122 may be set up in multiple modes, including a primary mode (may be referred to as a “master” mode when referring to a master/slave communication protocol). The MCU 122 ensures that all downstream components (i.e., distal components including the image sensors 124, which may be referred to as “slaves” in the master/slave communication protocol) are apprised of the configurations for upcoming data frames. The upcoming configurations may include, for example, gain, exposure duration, readout duration, pixel binning configuration, and so forth.

The MCU 122 includes internal logic for executing triggers to coordinate different devices, including, for example multiple image sensors 124. The MCU 122 provides instructions for upcoming frames and executes triggers to ensure that each image sensor 124 begins to capture data the same time. In some cases, the image sensors 124 may automatically advance to a subsequent data frame without receiving a unique trigger from the MCU 122.

In some cases, the endoscope 110 includes two or more image sensors 124 that detect EMR and output data frames simultaneously. The simultaneous data frames may be used to output a three-dimensional image and/or output imagery with increased definition and dynamic range. The pixel array of the image sensor 124 may include active pixels and optical black (“OB”) or optically blind pixels. The optical black pixels may be read during a blanking period of the pixel array when the pixel array is “reset” or calibrated. After the optical black pixels have been read, the active pixels are read during a readout period of the pixel array. The active pixels accumulate EMR that is pulsed by the emitter 102 during the blanking period of the image sensor 124. The pixel array 125 may include monochromatic or “color agnostic” pixels that do not comprise any filter for selectively receiving certain wavebands of EMR. The pixel array may include a color filter array (CFA), such as a Bayer pattern CFA, that selectively allows certain wavebands of EMR to pass through the filters and be accumulated by the pixel array.

The image sensor 124 is instructed by a combination of the MCU 122 and the controller 104 working in a coordinated effort. Ultimately, the MCU 122 provides the image sensor 124 with instructions on how to capture the upcoming data frame. These instructions include, for example, an indication of the gain, exposure, white balance, exposure duration, readout duration, pixel binning configuration, and so forth for the upcoming data frame. When the image sensor 124 is reading out data for a current data frame, the MCU 122 is rewriting the correct registers for the next data frame. The MCU 122 and the image sensor 124 operate in a back-and-forth data flow, wherein the image sensor 124 provides data to the MCU 122 and the MCU 122 rewrites correct registers to the image sensor 124 for each upcoming data frame. The MCU 122 and the image sensor 124 may operate according to a “ping pong buffer” in some configurations.

The image sensor 124, MCU 122, and controller 104 engage in a feedback loop to continuously adjust and optimize configurations for upcoming data frames based on output data. The MCU 122 continually rewrites correct registers to the image sensor 124 depending on the type of upcoming data frame (i.e., color data frame, multispectral data frame, fluorescence data frame, topographical mapping data frame, and so forth), configurations for previously output data frames, and user input. In an example implementation, the image sensor 124 outputs a multispectral data frame in response to the emitter 102 pulsing a multispectral waveband of EMR. The MCU 122 and/or controller 104 determines that the multispectral data frame is underexposed and cannot successfully be analyzed by a corresponding machine learning algorithm. The MCU 122 and/or controller 104 than adjusts configurations for upcoming multispectral data frames to ensure that future multispectral data frames are properly exposed. The MCU 122 and/or controller 104 may indicate that the gain, exposure duration, pixel binning configuration, etc. must be adjusted for future multispectral data frames to ensure proper exposure. All image sensor 124 configurations may be adjusted in real-time based on previously output data processed through the feedback loop, and further based on user input.

The waveguides 130, 131 include one or more optical fibers. The optical fibers may be made of a low-cost material, such as plastic to allow for disposal of one or more of the waveguides 130, 131. In some implementations, one or more of the waveguides 130, 131 include a single glass fiber. In some implementations, one or more of the waveguides 130, 131 include a plurality of glass fibers.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of white EMR. In an alternative example, the desired visualization scheme may indicate the user wishes to be notified when nerve tissue can be identified in the scene and/or when a tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the tool is within the threshold distance from the nerve tissue. The controller 104 may reconfigure the variable pulse cycle in real-time in response to receiving a revised desired visualization scheme from the user.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of broad spectrum visible EMR. In an alternative example, the desired visualization scheme may indicate the user wishes to be notified when nerve tissue can be identified in the scene and/or when a tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the tool is within the threshold distance from the nerve tissue. The controller 104 may reconfigure the variable pulse cycle in real-time in response to receiving a revised desired visualization scheme from the user.

FIG. 2A illustrates wherein the emitter cycles one or more EMR sources on and off to emit a pulse of EMR during each of a plurality of separate blanking periods of the pixel array 125. Specifically, the emitter 102 emits pulsed EMR during each of a T1 blanking period, T2 blanking period, T3 blanking period, and T4 blanking period of the pixel array 125. The pixel array 125 accumulates EMR during its blanking periods and reads out data during its readout periods.

Specifically, the pixel array 125 accumulates EMR during the T1 blanking period and reads out the T1 data frame during the T1 readout period, which follows the T1 blanking period. Similarly, the pixel array 125 accumulates EMR during the T2 blanking period and reads out the T2 data frame during the T2 readout period, which follows the T2 blanking period. The pixel array 125 accumulates EMR during the T3 blanking period and reads out the T3 data frame during the T3 readout period, which follows the T3 blanking period. The pixel array 125 accumulates EMR during the T4 blanking period and reads out the T4 data frame during the T4 readout period, which follows the T4 blanking period. Each of the T1 data frame, the T2 data frame, the T3 data frame, and the T4 data frame is provided to the image signal processor 140.

The contents of each of the T1-T4 data frames is dependent on the type of EMR that was pulsed by the emitter 102 during the preceding blanking period. For example, if the emitter 102 pulses white light during the preceding blanking period, then the resultant data frame may include a color data frame (if the pixel array 125 includes a color filter array for outputting red, green, and blue image data). Further for example, if the emitter 102 pulses a multispectral waveband of EMR during the preceding blanking period, then the resultant data frame is a multispectral data frame comprising information for identifying a spectral response by one or more objects within the scene and/or information for “seeing through” one or more structures within the scene. Further for example, if the emitter 102 pulses a fluorescence excitation waveband of EMR during the preceding blanking period, then the resultant data frame is a fluorescence data frame comprising information for identifying a fluorescent reagent or autofluorescence response by a tissue within the scene. Further for example, if the emitter 102 pulses EMR in a mapping pattern during the preceding blanking period, then the resultant data frame is a mapping data frame comprising information for calculating one or more of a three-dimensional topographical map of the scene, a dimension of one or more objects within the scene, a distance between two or more objects within the scene, and so forth.

Some “machine vision” or “computer vision” data frames, including multispectral data frames, fluorescence data frames, and mapping data frames may be provided to a corresponding algorithm or neural network configured to evaluate the information therein. A multispectral algorithm may be configured to identify one or more tissue structures within a scene based on how those tissue structures respond to one or more different wavebands of EMR selected for multispectral imaging. A fluorescence algorithm may be configured to identify a location of a fluorescent reagent or auto-fluorescing tissue structure within a scene. A mapping algorithm may be configured to calculate one or more of a three-dimensional topographical map of a scene, a depth map, a dimension of one or more objects within the scene, and/or a distance between two or more objects within the scene based on the mapping data frame.

FIG. 2B illustrates an example wherein the emitter 102 cycles separate visible, multispectral, fluorescence, and mapping EMR sources to emit pulsed visible 204, pulsed multispectral 206, pulsed fluorescence 208, and pulsed EMR in a mapping pattern 210. It should be appreciated that FIG. 2B is illustrative only, and that the emissions 204, 206, 208, 210 may be emitted in any order, may be emitted during a single visualization session as shown in FIG. 2B, and may be emitted during separate visualization sessions.

The pixel array 125 reads out a color data frame 205 in response to the emitter 102 pulsing the pulsed visible 204 EMR. The pulsed visible 204 EMR may specifically include a pulse of white light. The pixel array 125 reads out a multispectral data frame 207 in response to the emitter 102 pulsing the multispectral 206 waveband of EMR. It will be appreciated that the pulsed multispectral 206 waveband of EMR may include various wavebands used to elicit a spectral response. The pixel array 125 reads out a fluorescence data frame 209 in response to the emitter 102 pulsing the fluorescence 208 waveband of EMR. The pulsed fluorescence 208 waveband of EMR may specifically include one or more of EMR within a waveband from about 770-795 nm and/or 790-815 nm. The pixel array 125 reads out a mapping data frame 211 in response to the emitter 102 pulsing EMR in a mapping pattern 210. The pulsed mapping pattern 210 may include one or more of vertical hashing, horizontal hashing, a pin grid array, a dot array, a raster grid of discrete points, and so forth. Each of the color data frame 205, the multispectral data frame 207, the fluorescence data frame 209, and the mapping data frame 211 is provided to the image signal processor 140.

In an implementation, the emitter 102 separately pulses red, green, and blue visible EMR. In this implementation, the pixel array 125 may include a monochromatic (color agnostic) array of pixels. The pixel array 125 may separately read out a red data frame, a green data frame, and a blue data frame in response to the separate pulses of red, green, and blue visible EMR.

In an implementation, the emitter 102 separately pulses wavebands of visible EMR that are selected for capturing luminance (“Y”) imaging data, red chrominance (“Cr”) imaging data, and blue chrominance (“Cb”) imaging data. In this implementation, the pixel array 125 may separately read out a luminance data frame (comprising only luminance imaging information), a red chrominance data frame, and a blue chrominance data frame.

FIG. 2C illustrates a schematic flow chart diagram of a process flow for synchronizing operations of the emitter 102 and the pixel array 125. The process flow corresponds with the schematic diagram illustrated in FIG. 2A. The process flow includes the controller 104 instructing the emitter 102 to pulse EMR during a T1 blanking period of the pixel array 125 and then instructing the pixel array 125 to read out data during a T1 readout period following the T1 blanking period. Similarly, the controller 104 instructs the emitter to pulse EMR during each of the T2 blanking period, the T3 blanking period, and the T4 blanking period. The controller 104 instructs the emitter to read out data during each of the T2 readout period, the T3 readout period, and the T4 readout period that follow the corresponding blanking periods. Each of the output data frames are provided to the image signal processor 140.

The emitter 102 pulses according to a variable pulse cycle that includes one or more types of EMR. The variable pulse cycle may include visible EMR, which may include a white light emission, red light emission, green light emission, blue light emission, or some other waveband of visible EMR. The white light emission may be pulsed with a white light emitting diode (LED) or other light source and may alternatively be pulsed with a combination of red, green, and blue light sources pulsing in concert. The variable pulse cycle may include one or more wavebands of EMR that are selected for multispectral imaging or fluorescence imaging. The variable pulse cycle may include one or more emissions of EMR in a mapping pattern selected for three-dimensional topographical mapping or calculating dimensions within a scene. In some cases, several types of EMR are represented in the variable pulse cycle with different regularity than other types of EMR. This may be implemented to emphasize and de-emphasize aspects of the recorded scene as desired by the user.

The controller 104 adjusts the variable pulse cycle in real-time based on the visualization objectives. The system enables a user to input one or more visualization objectives and to change those objectives while using the system. For example, the visualization objective may indicate the user wishes to view only color imaging data, and in this case, the variable pulse cycle may include pulsed or constant emissions of white light (or other visible EMR). The visualization objective may indicate the user wishes to be notified when a scene includes one or more types of tissue or conditions that may be identified using one or more of color imaging, multispectral imaging, or fluorescence imaging. The visualization objective may indicate that a patient has been administered a certain fluorescent reagent or dye, and that fluorescence imaging should continue while the reagent or dye remains active. The visualization objective may indicate the user wishes to view a three-dimensional topographical map of a scene, receive information regarding distances or dimensions within the scene, receive an alert when a tool comes within critical distance from a certain tissue structure, and so forth.

The variable pulse cycle may include one or more finely tuned partitions of the electromagnetic spectrum that are selected to elicit a fluorescence response from a reagent, dye, or auto-fluorescing tissue. The fluorescence excitation wavebands of EMR include one or more of the following: 400±50 nm, 450±50 nm, 500±50 nm, 550±50 nm, 600±50 nm, 650±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, or 900±50 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

The variable pulse cycle may include one or more wavebands of EMR that are tuned for multispectral imaging. These wavebands of EMR are selected to elicit a spectral response from a certain tissue or penetrate through a certain tissue (such that substances disposed behind that tissue may be visualized). The multispectral wavebands of EMR include one or more of the following: 400±50 nm, 410±50 nm, 420±50 nm, 430±50 nm, 440±50 nm, 450±50 nm, 460±50 nm, 470±50 nm, 480±50 nm, 490±50 nm, 500±50 nm, 546±50 nm, 520±50 nm, 530±50 nm, 540±50 nm, 550±50 nm, 560±50 nm, 570±50 nm, 580±50 nm, 590±50 nm, 600±50 nm, 630b±50 nm, 620±50 nm, 630±50 nm, 640±50 nm, 650±50 nm, 660±50 nm, 670±50 nm, 680±50 nm, 690±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, 900±50 nm, 910±50 nm, 920±50 nm, 930±50 nm, 940±50 nm, 950±50 nm, 960±50 nm, 970±50 nm, 980±50 nm, 990±50 nm, 1000±50 nm, 900±100 nm, 950±100 nm, or 1000±100 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

Certain multispectral wavelengths pierce through tissue and enable a medical practitioner to “see through” tissues in the foreground to identify chemical processes, structures, compounds, biological processes, and so forth that are located behind the foreground tissues. The multispectral wavelengths may be specifically selected to identify a specific disease, tissue condition, biological process, chemical process, type of tissue, and so forth that is known to have a certain spectral response.

The variable pulse cycle may include one or more emissions of EMR that are optimized for mapping imaging, which includes, for example, three-dimensional topographical mapping, depth map generation, calculating distances between objects within a scene, calculating dimensions of objects within a scene, determining whether a tool or other object approaches a threshold distance from another object, and so forth. The pulses for laser mapping imaging include EMR formed in a mapping pattern, which may include one or more of vertical hashing, horizontal hashing, a dot array, and so forth.

The controller 104 optimizes the variable pulse cycle to accommodate various imaging and video standards. In most use-cases, the system outputs a video stream comprising at least 30 frames per second (fps). The controller 104 synchronizes operations of the emitter and the image sensor to output data at a sufficient frame rate for visualizing the scene and further for processing the scene with one or more advanced visualization techniques. A user may request a real-time color video stream of the scene and may further request information based on one or more of multispectral imaging, fluorescence imaging, or laser mapping imaging (which may include topographical mapping, calculating dimensions and distances, and so forth). The controller 104 causes the image sensor to separately sense color data frames, multispectral data frames, fluorescence data frames, and mapping data frames based on the variable pulse cycle of the emitter.

In some cases, a user requests more data types than the system can accommodate while maintaining a smooth video frame rate. The system is constrained by the image sensor's ability to accumulate a sufficient amount of electromagnetic energy during each blanking period to output a data frame with sufficient exposure. In some cases, the image sensor outputs data at a rate of 60-120 fps and may specifically output data at a rate of 60 fps. In these cases, for example, the controller 104 may devote 24-30 fps to color visualization and may devote the other frames per second to one or more advanced visualization techniques.

The controller 104 calculates and adjusts the variable pulse cycle of the emitter 102 in real-time based at least in part on the known capabilities of the pixel array 125. The controller 104 may access data stored in memory indicating how long the pixel array 125 must be exposed to a certain waveband of EMR for the pixel array 125 to accumulate a sufficient amount of EMR to output a data frame with sufficient exposure. In most cases, the pixel array 125 is inherently more or less sensitive to different wavebands of EMR. Thus, the pixel array 125 may require a longer or shorter blanking period duration for some wavebands of EMR to ensure that all data frames output by the image sensor 124 comprise sufficient exposure levels.

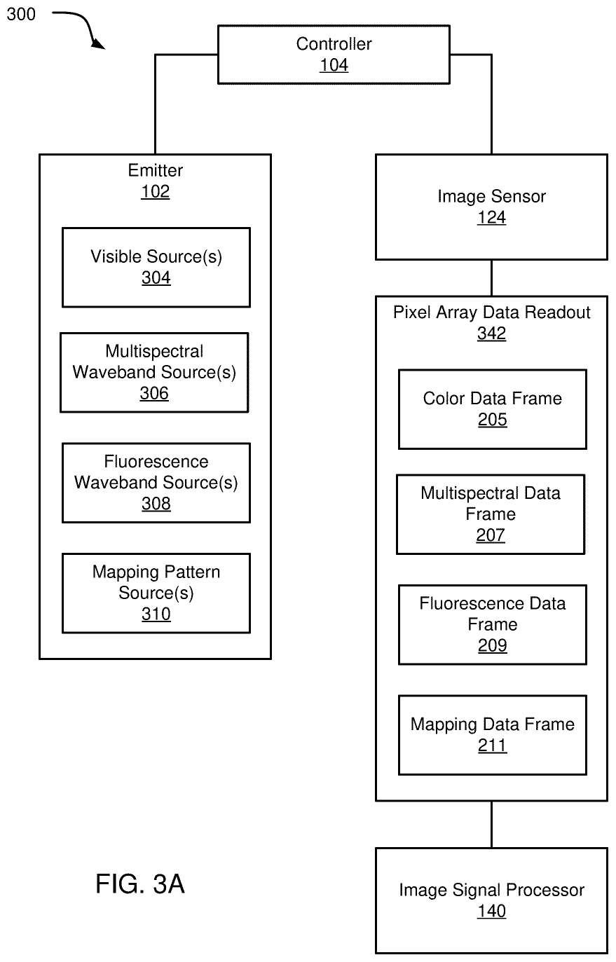

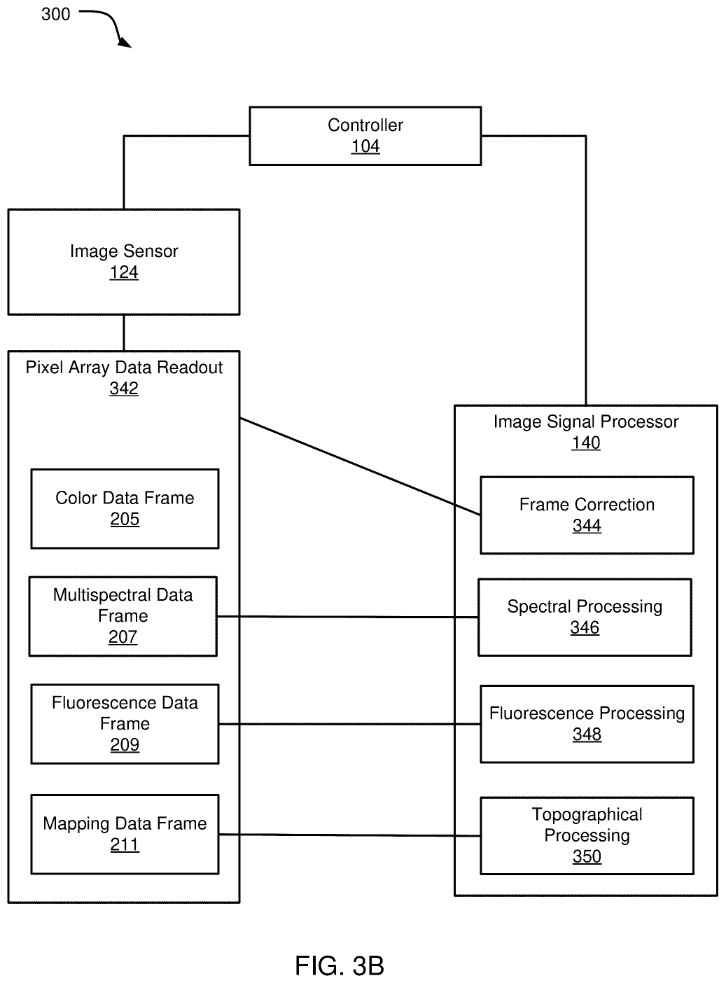

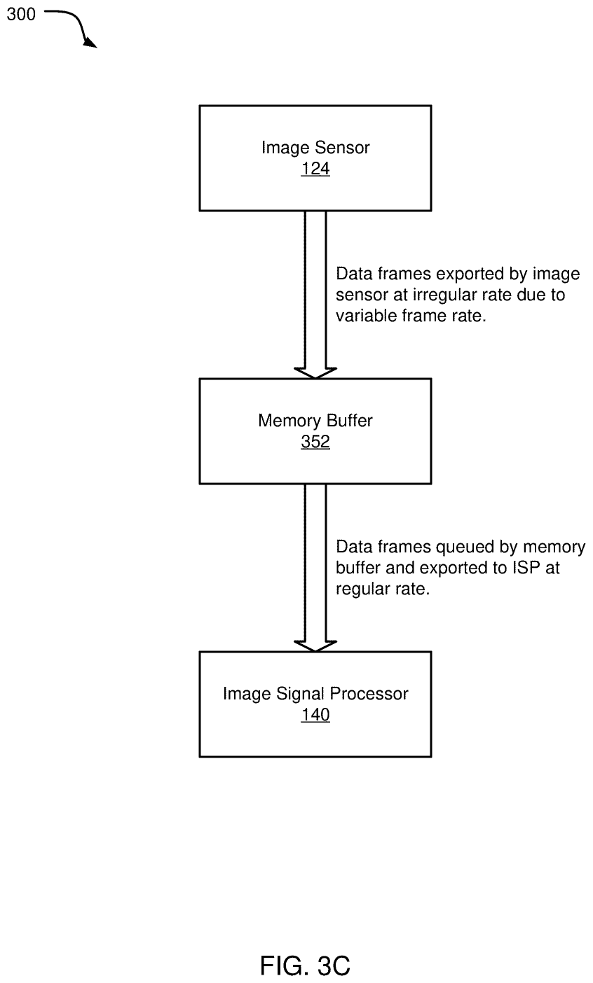

The controller 104 determines the data input requirements for various advanced visualization algorithms (see, e.g., the algorithms 346, 348, 350 first described in FIG. 3B). For example, the controller 104 may determine that certain advanced visualization algorithms do not require a data input at the same regularity as a color video stream output of 30 fps. In these cases, the controller 104 may optimize the variable pulse cycle to include white light pulses at a more frequent rate than pulses for advanced visualization such as multispectral, fluorescence, or laser mapping imaging. Additionally, the controller 104 determines whether certain algorithms may operate with lower resolution data frames that are read out by the image sensor using a pixel binning configuration. In some cases, the controller 104 ensures that all color frames provided to a user are read out in high-resolution (without pixel binning). However, some advanced visualization algorithms (see e.g., 346, 348, 350) may execute with lower resolution data frames.

The system 100 may include a plurality of image sensors 124 that may have different or identical pixel array configurations. For example, one image sensor 124 may include a monochromatic or “color agnostic” pixel array with no filters, another image sensor 124 may include a pixel array with a Bayer pattern CFA, and another image sensor 124 may include a pixel array with a different CFA. The multiple image sensors 124 may be assigned to detect EMR for a certain imaging modality, such as color imaging, multispectral imaging, fluorescence imaging, or laser mapping imaging. Further, each of the image sensors 124 may be configured to simultaneously accumulate EMR and output a data frame, such that all image sensors are capable of sensing data for all imaging modalities.

The controller 104 prioritizes certain advanced visualization techniques based on the user's ultimate goals. In some cases, the controller 104 prioritizes outputting a smooth and high-definition color video stream to the user above other advanced visualization techniques. In other cases, the controller 104 prioritizes one or more advanced visualization techniques over color visualization, and in these cases, the output color video stream may appear choppy to a human eye because the system outputs fewer than 30 fps of color imaging data.

For example, a user may indicate that a fluorescent reagent has been administered to a patient. If the fluorescent reagent is time sensitive, then the controller 104 may ensure that a sufficient ratio of frames is devoted to fluorescence imaging to ensure the user receives adequate fluorescence imaging data while the reagent remains active. In another example, a user requests a notification whenever the user's tool comes within a threshold distance of a certain tissue, such as a blood vessel, nerve fiber, cancer tissue, and so forth. In this example, the controller 104 may prioritize laser mapping visualization to constantly determine the distance between the user's tool and the surrounding structures and may further prioritize multispectral or fluorescence imaging that enables the system to identify the certain tissue. The controller 104 may further prioritize color visualization to ensure the user continues to view a color video stream of the scene.

FIGS. 3A-3C illustrate schematic diagrams of a system 300 for processing data output by an image sensor 124 comprising the pixel array 125. The system 300 includes a controller 104 in communication with each of the emitter 102 and the image sensor 124 comprising the pixel array 125. The emitter 102 includes one or more visible sources 304, multispectral waveband sources 306, fluorescence waveband sources 308, and mapping pattern sources 310 of EMR. The pixel array data readout 342 of the image sensor 124 includes one or more of the color data frames 205, multispectral data frames 207, fluorescence data frames 209, and mapping data frames 211 as discussed in connection with FIG. 2B.

As illustrated in FIG. 3B, all data read out by the pixel array may undergo frame correction 344 processing by the image signal processor 140. In various implementations, one or more of the color data frame 205, the multispectral data frame 207, the fluorescence data frame 209, and the mapping data frame 211 undergoes frame correction 344 processes. The frame correction 344 includes one or more of sensor correction, white balance, color correction, or edge enhancement.

The multispectral data frame 207 may undergo spectral processing 346 that is executed by the image signal processor 140 and/or another processor that is external to the system 300. The spectral processing 346 may include a machine learning algorithm and may be executed by a neural network configured to process the multispectral data frame 207 to identify one or more tissue structures within a scene based on whether those tissue structures emitted a spectral response.

The fluorescence data frame 209 may undergo fluorescence processing 348 that is executed by the image signal processor 140 and/or another processor that is external to the system 300. The fluorescence processing 348 may include a machine learning algorithm and may be executed by a neural network configured to process to fluorescence data frame 209 and identify an intensity map wherein a fluorescence relaxation wavelength is detected by the pixel array.

The mapping data frame 211 may undergo topographical processing 350 that is executed by the image signal processor 140 and/or another processor that is external to the system 300. The topographical processing 350 may include a machine learning algorithm and may be executed by a neural network configured to assess time-of-flight information to calculate a depth map representative of the scene. The topographical processing 350 includes calculating one or more of a three-dimensional topographical map of the scene, a dimension of one or more objects within the scene, a distance between two or more objects within the scene, a distance between a tool and a certain tissue structure within the scene, and so forth.

FIG. 3C illustrates a schematic diagram of a system 300 and process flow for managing data output at an irregular rate. The image sensor 124 operates according to a sensor cycle that includes blanking periods and readout periods. The image sensor 124 outputs a data frame at the conclusion of each readout period that includes an indication of the amount of EMR the pixel array accumulated during the preceding accumulation period or blanking period.

Each frame period in the sensor cycle is adjustable on a frame-by-frame basis to optimize the output of the image sensor and compensate for the pixel array 125 having varying degrees of sensitivity to different wavebands of EMR. The duration of each blanking period may be shortened or lengthened to customize the amount of EMR the pixel array 125 can accumulate. Additionally, the duration of each readout period may be shortened or lengthened by implementing a pixel binning configuration or causing the image sensor to read out each pixel within the pixel array 125. Thus, the image sensor 124 may output data frames at an irregular rate due to the sensor cycle comprising a variable frame rate. The system 300 includes a memory buffer 352 that receives data frames from the image sensor 124. The memory buffer 352 stores the data frames and then outputs each data frame to the image signal processor 140 at a regular rate. This enables the image signal processor 140 to process each data frame in sequence at a regular rate.

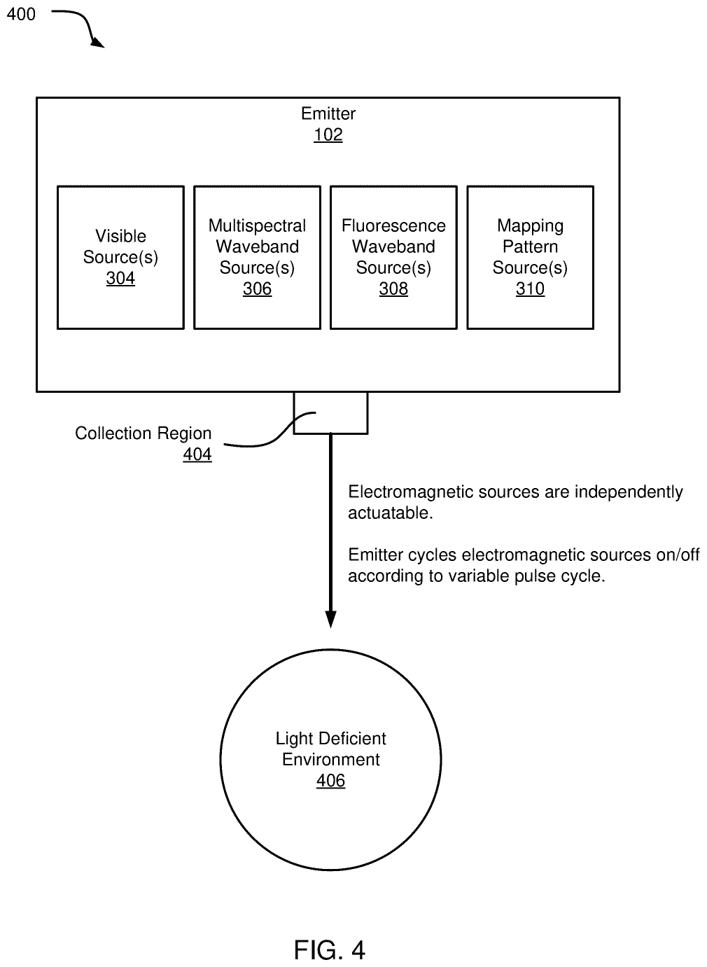

FIG. 4 is a schematic diagram of an illumination system 400 for illuminating a light deficient environment 406 such as an interior of a body cavity. In most cases, the emitter 102 is the only source of illumination within the light deficient environment 406 such that the pixel array of the image sensor does not detect any ambient light sources. The emitter 102 includes a plurality of separate and independently actuatable sources of EMR, which may include visible source(s) 304, multispectral waveband source(s) 306, fluorescence waveband source(s) 308, and mapping pattern source(s) 310. The emitter may cycle a selection of the sources on and off to pulse according to the variable pulse cycle received from the controller 104. Each of the EMR sources feeds into a collection region 404 of the emitter 102. The collection region 404 may then feed into a waveguide (see e.g., 130 in FIG. 1A) that transmits the pulsed EMR to a distal end of an endoscope within the light deficient environment 406.

The variable pulsing cycle is customizable and adjustable in real-time based on user input. The emitter 102 may instruct the individual EMR sources to pulse in any order. Additionally, the emitter 102 may adjust one or more of a duration or an intensity of each pulse of EMR. The variable pulse cycle may be optimized to sufficiently illuminate the light deficient environment 406 such that the resultant data frames read out by the pixel array 125 are within a desired exposure range (i.e., the frames are neither underexposed nor overexposed). The desired exposure range may be determined based on user input, requirements of the image signal processor 140, and/or requirements of a certain image processing algorithm (see 344, 346, 348, and 350 in FIG. 3B). The sufficient illumination of the light deficient environment 406 is dependent on the energy output of the individual EMR sources and is further dependent on the efficiency of the pixel array 125 for sensing different wavebands of EMR.

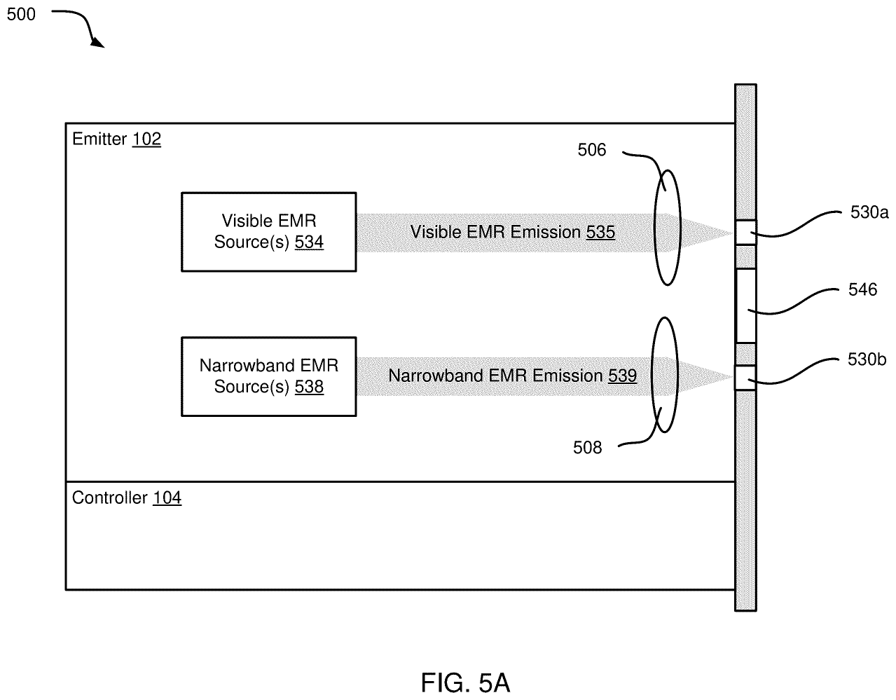

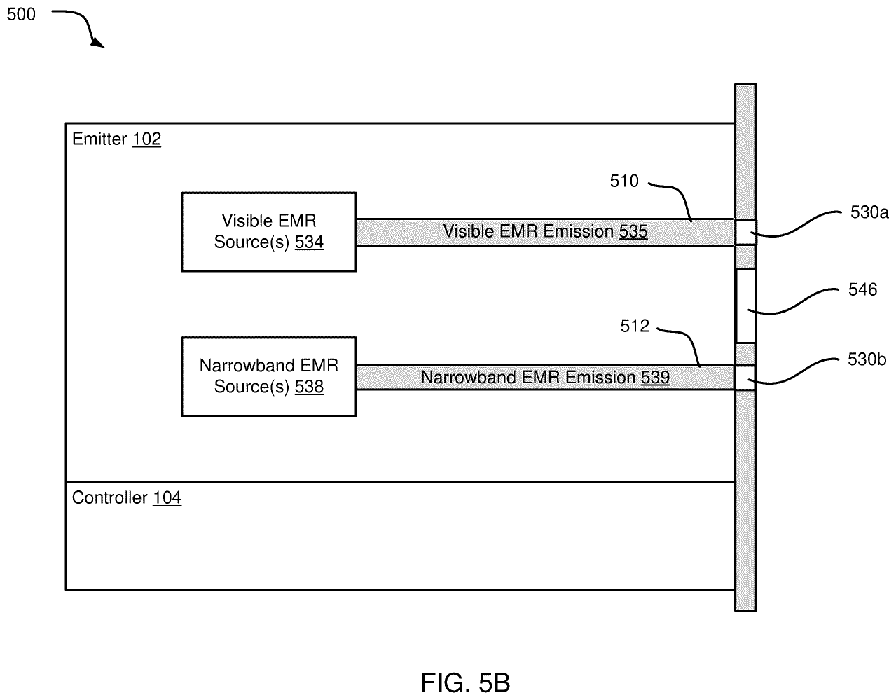

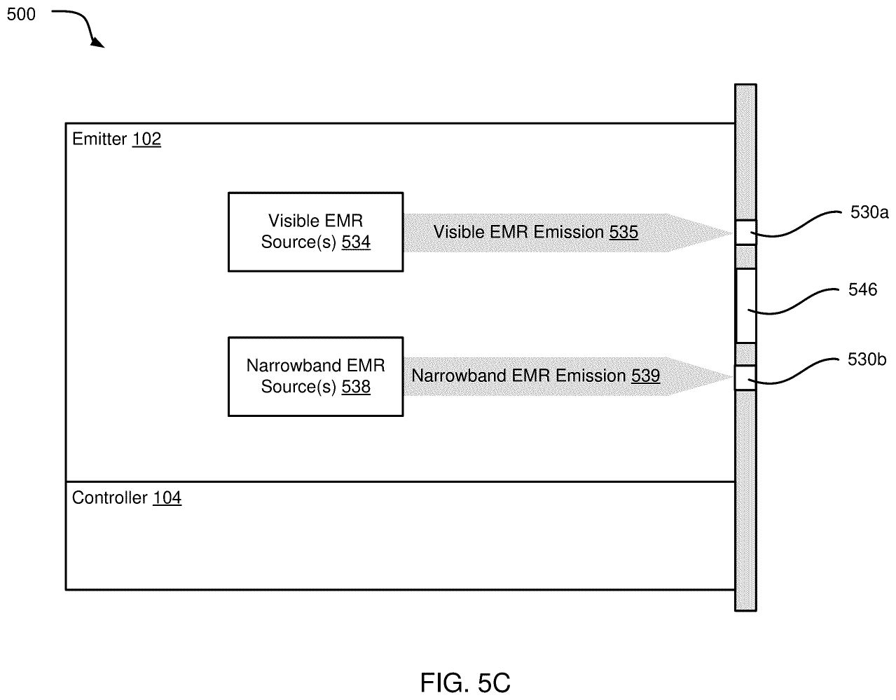

FIGS. 5A-5C are a schematic illustrations of varying implementations of a control unit 500 for illumination and visualization for an endoscopic visualization system. Specifically, FIG. 5A is a schematic illustration of a control unit 500 including lenses for focusing EMR, FIG. 5B is a schematic illustration of a control unit 500 including fiber optic bundles for transmitting EMR, and FIG. 5C is a schematic illustration of a control unit 500 configured for directed transmission of EMR into a waveguide contact. Any of the embodiments illustrated in FIGS. 5A-5C may be implemented in connection with any of the systems and methods described herein. The control units 500 illustrated in FIGS. 5A-5C may include each of the components discussed in connection with the emitter 102 and the controller 104 first discussed in connection with FIGS. 1A-1C.

The control unit 500 includes one or more visible EMR sources 534, such as the white EMR source 134 first discussed in connection with FIG. 1C. The control unit 500 includes one or more narrowband EMR sources 538, such as the EMR sources 138 first discussed in connection with FIG. 1C. The control unit 500 further include a data port 546 configured to interface with a corresponding data cable that transmits data from the control unit 500 to an endoscope. The data port 546 is a component of the data transmission pipeline 146 first discussed in connection with FIG. 1C. The data port 546 enables bidirectional communications between the endoscope 110 and the controller 104. The data port 546 may further enable bidirectional communications between the tool 108 and the controller 104.

The visible EMR source 534 pulses a visible EMR emission 535 that passes through a lens 506 configured to focus the visible EMR emission 535 into a collection region that feeds into a first waveguide contact 530a. The first waveguide contact 530a provides a means for a corresponding fiber optic waveguide, such as the first waveguide 130a discussed in connection with FIG. 1C, to interface with the emitter 102 and receive the visible EMR emission 535. The first waveguide contact 530a is dedicated to carrying the visible EMR emission 535 and providing only the visible EMR emission 535 to the corresponding fiber optic waveguide.

The one or more narrowband EMR sources 538 each pulse a narrowband EMR emission 539 that passes through a lens 508 configured to focus the narrowband EMR emission 539 into a collection region that feeds into a second waveguide contact 530b. The second waveguide contact 530b provides a means for a corresponding fiber optic waveguide, such as the second waveguide 130b discussed in connection with FIG. 1C, to interface with the emitter 102 and receive the narrowband EMR emission 539. The second waveguide contact 530b is dedicated to carrying the one or more narrowband EMR emissions 539 and providing only the one or more narrowband EMR emissions 539 to the corresponding fiber optic waveguide.

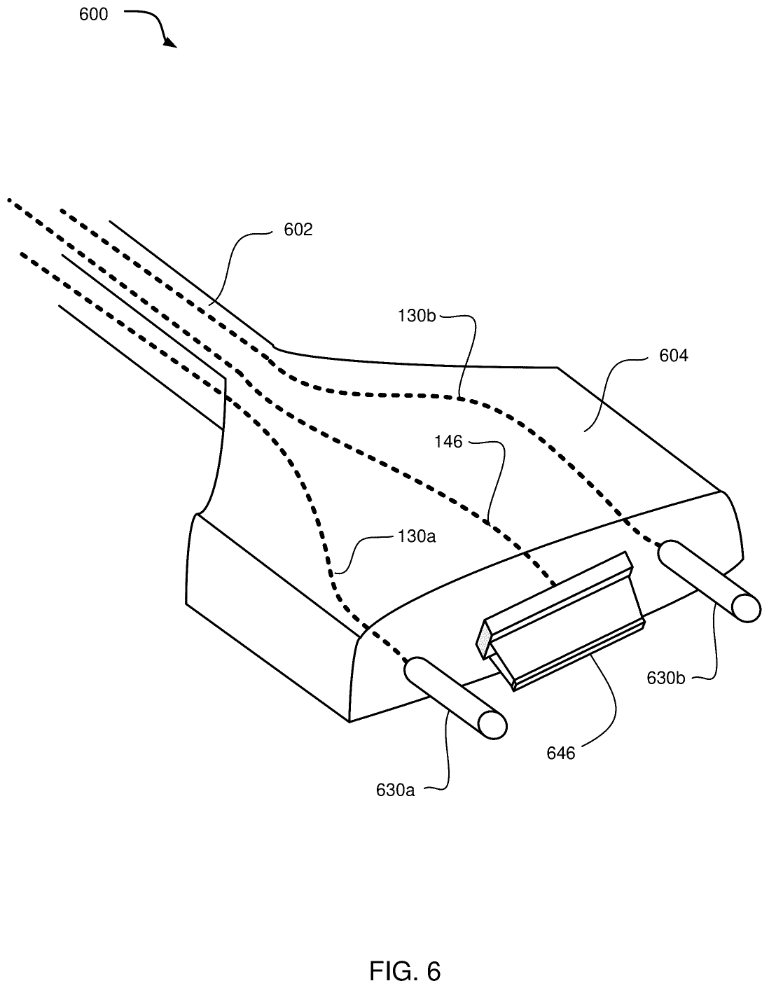

In an implementation, the waveguide contacts 530a, 530b are implemented as female contact openings that corresponds with male ferrule contacts on a fiber optic cable connection, as discussed in FIG. 6. In alternate implementations, the waveguide contacts 530a, 530b are be implemented as male ferrule contacts configured to interface with corresponding female contact openings disposed within the fiber optic cable connection.

FIG. 5B is a schematic illustration of an implementation wherein the control unit 500 does not include lenses 506, 508 for focusing EMR. Instead, in the implementation depicted in FIG. 5B, the visible EMR source(s) 534 pulses the visible EMR emission 535 directly into a first fiber optic bundle 510 that carries the visible EMR emission 535 to the first waveguide contact 530a. Further, the narrowband EMR source(s) 538 pulse the narrowband EMR emission 539 directly into a second fiber optic bundle 512 that carries the narrowband EMR emission 539 to the second waveguide contact 530b.

FIG. 5C is a schematic illustration of an implementation wherein the control unit 500 includes neither the lenses 506, 508 for focusing EMR (as in FIG. 5A) nor the fiber optic bundles 510, 512 for transmitting EMR (as in FIG. 5B) to the waveguide contacts 530a, 530b. In the implementation depicted in FIG. 5C, the visible EMR source 534 pulses the visible EMR emission 535 directly into the first waveguide contact 530a, where it is then collected by a fiber optic bundle associated with the endoscope system by way of a corresponding ferrule (see first fiber optic ferrule 630a illustrated in FIG. 6). Similarly, the narrowband EMR source 538 pulses the narrowband EMR emission 539 directly into the second waveguide contact 530b, where it is then collected by a fiber optic bundle associated with the endoscope system by way of a corresponding ferrule (see second fiber optic ferrule 630b illustrated in FIG. 6). Thus, in the implementation illustrated in FIG. 5C, the EMR sources 534, 538 pulse EMR directly into the waveguide contacts 530a, 530b without the use of intervening optical components such as lenses of fiber optic bundles. In some cases, the implementation illustrated in FIG. 5C is the most efficient with the least amount of EMR loss.

FIG. 6 is a perspective view of an example integrated cable connector 600 that is optimized for luminous efficiency. The connector 600 includes a multiconductor 602 feeding into an overmold housing 604. The connector 600 includes a first fiber optic ferrule 630a and a second fiber optic ferrule 630b attached to the overmold housing 604. The connector 600 includes a data connector 646 attached to the overmold housing 604.

The first fiber optic ferrule 630a receives the visible EMR emission 535 by way of the first waveguide contact 530a. The first fiber optic ferrule 630a feeds the visible EMR emission 535 into the first waveguide 130a that carries the visible EMR emission 535 to a distal end of the endoscope 110. The second fiber optic ferrule 630b receives the narrowband EMR emissions 539 by way of the second waveguide contact 530b. The second fiber optic ferrule 630b feeds the narrowband EMR emissions 539 into the second waveguide 130b that carries the narrowband EMR emissions 539 to a distal end of the endoscope 110.

The data connector 646 interfaces with the corresponding data port 546 disposed within a housing of the emitter 102 or the controller 104. Each of the data connector 646 and the corresponding data port 546 may be constructed with standard or custom specifications for a data communication coupling. The data connector 646 receives bidirectional communications between the controller 104 and the endoscope 110 and may further receive communications between the emitter 102 and the endoscope 110. The data connector 646 feeds those data communications into the data transmission pipeline 146, which carries communications to one or more of the MCU 122 or the image sensors 124. The data connector 646 depicted in FIG. 6 includes a board-based connector, but it should be appreciated that the disclosure is not limited to board-based connectors. In an implementation, the data connector 646 is a pin-based connector.

The integrated cable connector 600 described herein is fitted with two or more fiber optic ferrules 630a, 630b for transmission of EMR. The emitter 102 includes separate and independently actuatable sources of EMR, which may include one or more of broadband light sources, LED sources, laser diode sources, and so forth. In most implementations, it is advantageous to separate emissions by the LED sources and the laser diode sources to maintain efficiency of the illumination output.

Each of the fiber optic ferrules 630a, 630b communicates with a dedicated bundle of optical fibers that travels the length of the multiconductor 602 and further travels a length of a lumen of the endoscope 110. The two or more dedicated bundles of optical fibers may be randomized within the multiconductor 602 cabling, and further within the lumen of the endoscope 110. This enables the optical fibers to be combined within the multiconductor 602 of the integrated cable connector 600 rather than within the emitter 102 itself. Thus, the optical fibers are mixed within the multiconductor 602 and the lumen of the endoscope and are then split again at the distal end of the lumen of the endoscope.

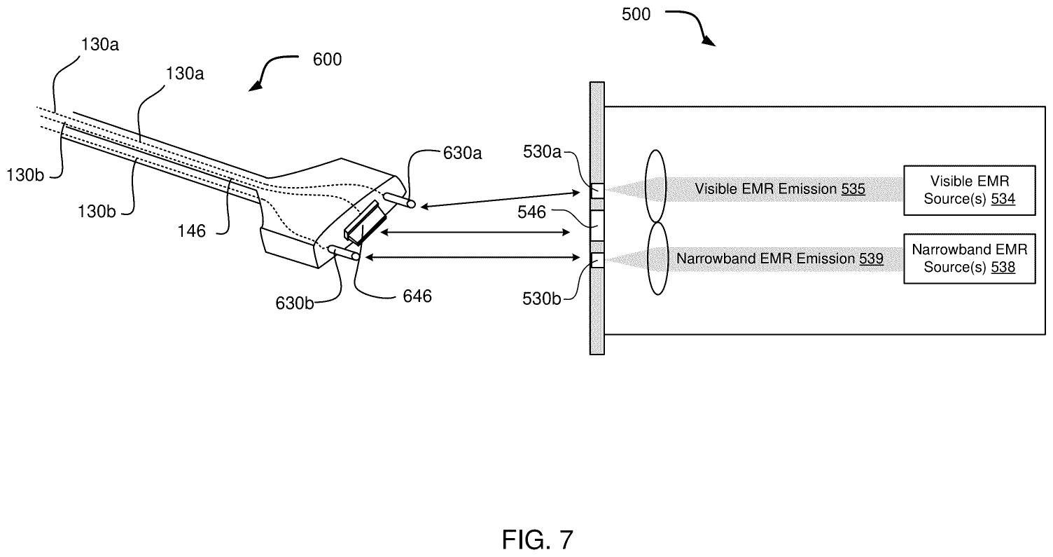

Each fiber optic ferrule 630a, 630b is capable of collecting white light, narrowband visible light, narrowband multispectral light, or narrowband fluorescence light. The fiber optic ferrules 630a, 630b become dedicated to a single type of EMR when the integrated cable connector 600 is plugged into the control unit 500, as shown in FIG. 7.

The integrated cable connector 600 enables highly efficient coupling of the different EMR sources 134, 138 within the emitter 102 to the separate fiber optic ferrules 630a, 630b. This enables the system 100 to provide sufficient illumination from lower power sources. The lower power sources may provide multiple advantages to the system 100, including reduced cost, reduced size, simplified thermal management, and reduced power consumption. Thus, it is advantageous to use lower power EMR sources when possible, and then maintain separation between those lower power EMR sources and the higher power EMR sources. This reduces illumination losses and illumination inefficiencies.

FIG. 7 is a schematic illustration of the integrated cable connector 600 interfacing with the control unit 500. The connector 600 is configured to plug into a corresponding port on the control unit 500 to receive illumination and data from the control unit 500.

The first fiber optic ferrule 630a receives the visible EMR emission 535 from the corresponding first waveguide contact 530a disposed within a housing of the control unit 500. The second fiber optic ferrule 630b receives the narrowband EMR emission 539 from the corresponding second waveguide contact 530b disposed within the housing of the control unit 500. The data connector 646 of the connector 600 interfaces with the corresponding data port 546 of the control unit 500.

The configuration illustrated in FIG. 7 enables increased luminous efficiency when compared with an alternative embodiment wherein EMR from the visible sources 534 and the narrowband EMR sources 538 are combined within the emitter 102 itself. If the visible EMR sources 534 and the narrowband EMR sources 538 were combined within the emitter 102, then the system 100 would experience significant light loss at each junction. The configuration illustrated in FIG. 7 prevents light loss and increases luminous efficiency of the system 100.

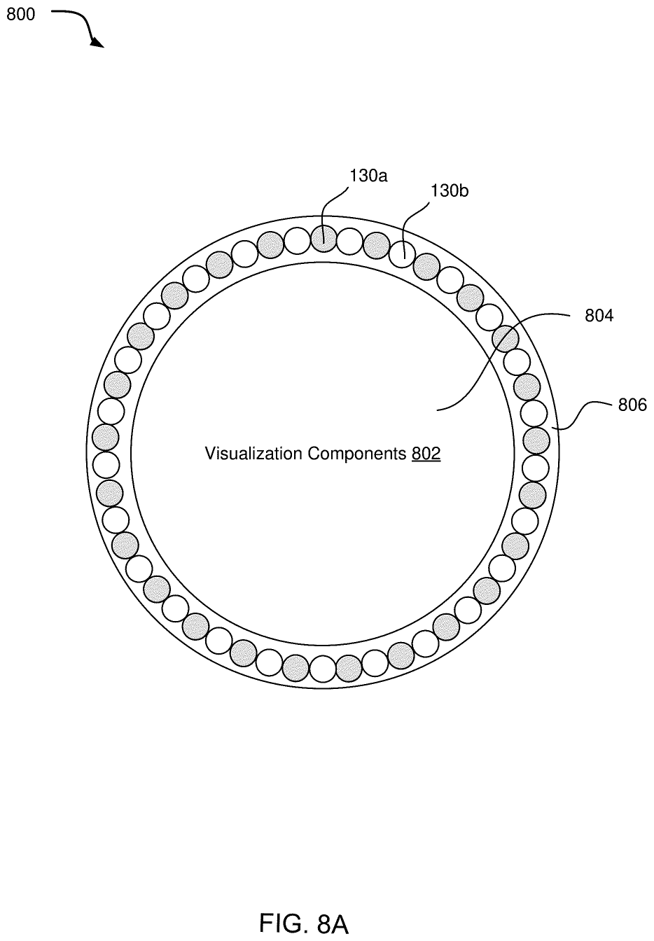

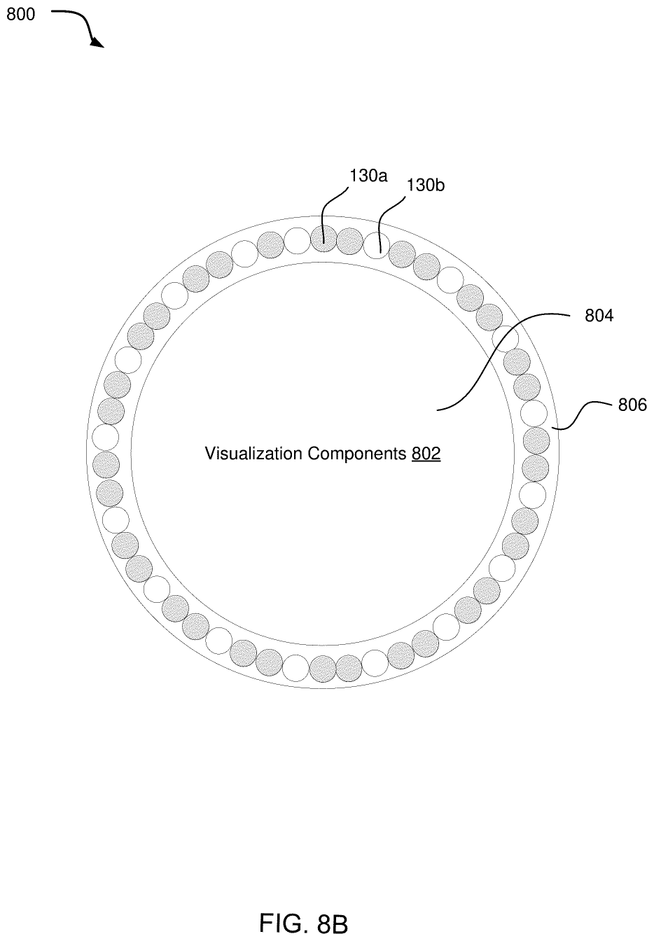

FIGS. 8A and 8B are schematic cross-sectional illustrations of a transverse section of a lumen of an endoscope 110. The cross-sectional views of the lumen illustrate implementations wherein visualization components 802, such as those illustrated and discussed in connection with FIG. 1B, are disposed within an interior region of the lumen, and optical fibers surround the visualization components 802. The individual optical fibers (depicted by the ring of circles shown in FIGS. 8A-8B) collectively make up the waveguide 130. The grey-shaded optical fibers represent the first waveguide 130a, and the white-shaded optical fibers represent the second waveguide 130b.

The schematic illustrations depicted in FIGS. 8A-8B show a single ring of optical fibers encircling the visualization components 802. However, it should be appreciated that the system may include many optical fibers, and in some cases may include hundreds or thousands of optical fibers. In actuality, the lumen of the endoscope 110 may be filled with multiple rows of optical fibers that may be pressed together in a tight configuration. It should be understood that the configurations illustrated in FIGS. 8A-8B are for discussion purposes only and should not be viewed as illustrative of an actual implementation.

In some implementations, the lumen of the endoscope includes two or more concentric channels or tubes running along a longitudinal length of the lumen. In the example implementations illustrated in FIGS. 8A and 8B, the lumen includes a first innermost channel 804 and a second channel 806 disposed around the first innermost channel 804. Thus, the first innermost channel 804 and the second channel 806 function as concentric tubes running a length of the lumen. The result is similar to the implementation depicted in FIG. 1B, wherein the waveguide 130 transmits the emissions of EMR 144 with uniformity around the visualization components 802, which includes the lenses 126, filters 128, prisms 132, and image sensors 124.

In alternative implementations, the lumen does not include concentric channels 804, 806 as shown in FIGS. 8A-8B. Instead, the plurality of optical fibers is still disposed around the visualization components 802. The plurality of optical fibers may be pressed against an interior surface of the lumen, such that the plurality of optical fibers encircles the visualization components 802.

The ratio of a quantity of the optical fibers making up the first waveguide 130a versus the quantity of the optical fibers making up the second waveguide 130b may be optimized based one or more factors. This ratio may be optimized based on the type of EMR intended to be transmitted through the first waveguide 130a or the second waveguide 130b. This ratio may be optimized based on a characteristic of the source of EMR that will pulse EMR into the first or second waveguide 130a, 130b, for example, the intensity of the source of EMR, the wavelengths intended to be pulsed by the source of EMR, the power provided to the source of EMR, and so forth. In some cases, additional optical fibers are dedicated to the waveguide 130 expected to transmit lower energy or dimmer EMR. Thus, the ratio may be optimized to ensure relatively consistent or uniform illumination of the scene regardless of whether the system is pulsing white light, narrowband EMR for multispectral visualization, narrowband EMR for fluorescence visualization, or mapping patterns for topographical mapping.

The ratio of the quantity of the optical fibers of the first waveguide 130a and the second waveguide 130b may be roughly equal to 1:1. In alternative implementations, the ratio may be about 1.25:1, 1.5:1, 2:1, 2.5:1, 3:1, 4:1, 5:1, and so forth depending on the types of EMR sources being used and the desired visualization characteristics.

Each of the first waveguide 130a and the second waveguide 130b is composed of numerous optical fibers. In some implementations, each waveguide 130a, 130b consists of hundreds or thousands of optical fibers. These individual optical fibers are physically intermixed as shown in FIGS. 8A and 8B but remain dedicated to transmitting a certain type of EMR. Thus, the optical fibers for the first waveguide 130a (depicted with grey shading in FIGS. 8A-8B) are physically intermixed with the optical fibers for the second waveguide 130b (depicted with white shading in FIGS. 8A-8B). This physical intermixing enables increased uniformity in the resultant emission of EMR when the endoscope 110 is in use. However, the optical fibers for the first waveguide 130a and the second waveguide 130b are not optically intermixed. Thus, the optical fibers for the first waveguide 130a remain dedicated to transmitting the visible EMR emissions 535, and the optical fibers for the second waveguide 130b remain dedicated to transmitting the narrowband EMR emissions 539.

FIG. 8A illustrates an example embodiment wherein the optical fibers are allocated to the first waveguide 130a (transmitting the visible EMR emission 535) and the second waveguide 130b (transmitting the narrowband EMR emission 539) with a 1:1 ratio, such that the first waveguide 130a and the second waveguide 130b comprise roughly equal quantities of optical fibers. FIG. 8B illustrates an alternative example embodiment wherein the optical fibers are allocated to the first waveguide 130a and the second waveguide 130b with a 2:1 ratio, such that the first waveguide 130a comprises roughly two times as many optical fibers as the second waveguide 130b.

The implementations illustrated in FIGS. 8A-8B are illustrative only, and it should be understood that the optical fibers may be allocated to the first waveguide 130a and the second waveguide 130b with varying ratios depending on the implementation. The quantity of optical fibers allocated to each of the first waveguide 130a and the second waveguide 130b may be optimized depending on the type of EMR being transmitted by the respective waveguides 130a, 130b. This optimization may be determined based on, for example, the amount of energy of the EMR, the waveband of the EMR, the frequency of the EMR, and so forth. The quantity of optical fibers may be further allocated based on the desired illumination of the scene. For example, in some cases it may be necessary to allocate additional optical fibers to carrying an emission by a white LED to ensure the scene is adequately illuminated for visualization.

In an example implementation, the combined waveguide 130 includes roughly 2,000 individual optical fibers. In this example, about 500 optical fibers are dedicated to transmitting the narrowband EMR emissions 538, and about 1,500 optical fibers are dedicated to transmitting the visible EMR emissions 535. The 2,000 optical fibers are intermixed within the lumen of the endoscope to ensure that the scene is uniformly illuminated with each of the visible EMR emissions 535 and the narrowband EMR emissions 539.

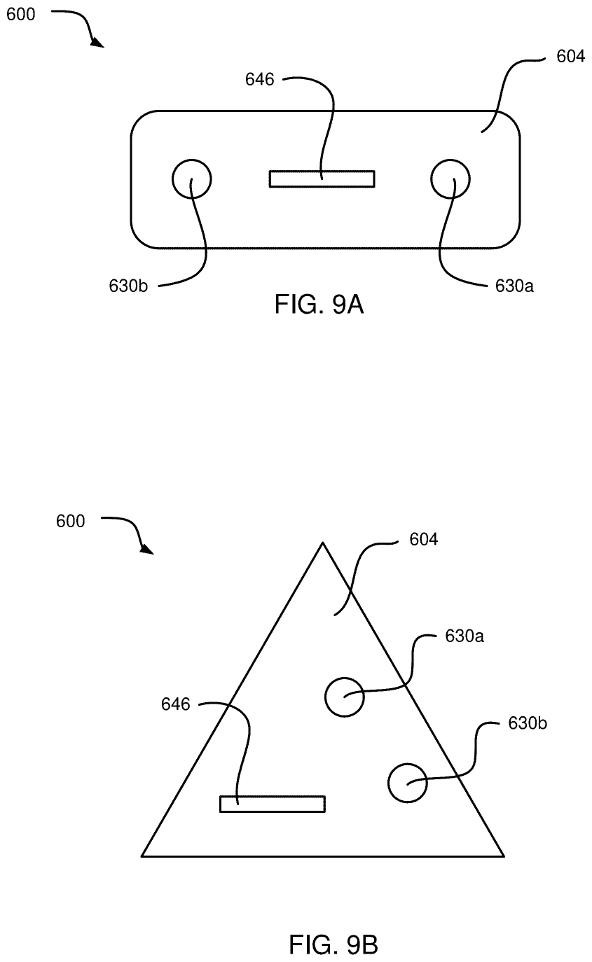

FIGS. 9A-9D illustrate straight-on views of a terminating end of example integrated cable connectors 600. The integrated cable connectors 600 include the overmold housing 604 with the data connector 646, first fiber optic ferrule 630a, and second fiber optic ferrule 630b disposed therein. FIGS. 9A-9D illustrate how the geometry of the overmold housing 604 and the configuration of the components 646, 630a, 630b may be adjusted depending on the implementation, without departing from the scope of this disclosure. It should be appreciated that the integrated cable connector 600 may have any suitable geometric configuration. In some implementations, the geometry of the overmold housing 604 is selected to ensure that the integrated cable connector 600 can only be oriented one way within a corresponding plug for the control unit 500. This prevents disorientation when plugging the endoscope into the control unit 500.

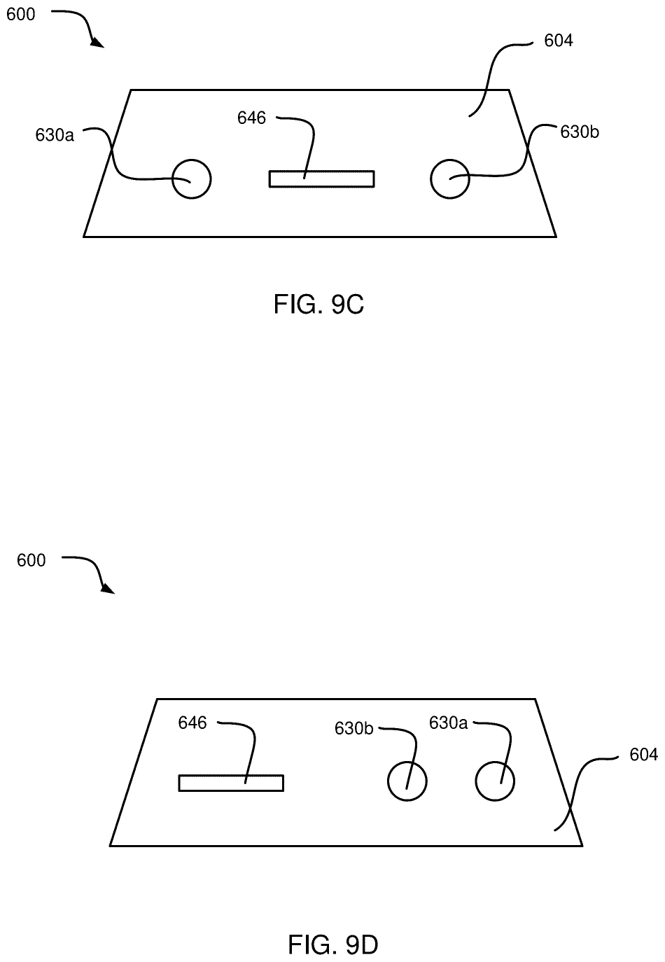

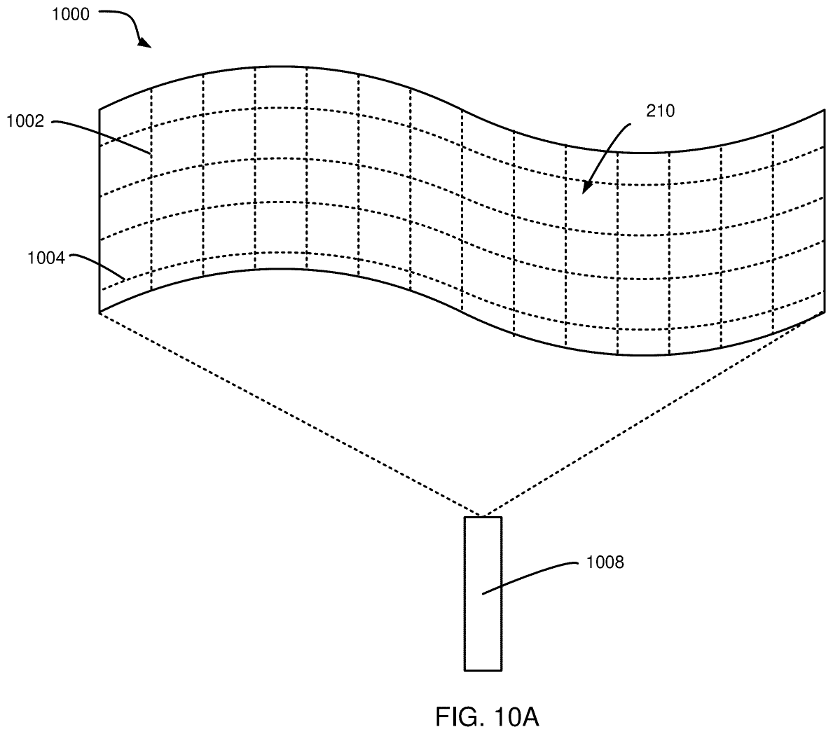

FIGS. 10A and 10B illustrate schematic diagrams of a system 1000 for emitting EMR in a mapping pattern 210. The emitter 102 may pulse the mapping pattern 210 using a low mode laser light that is diffracted to generate the applicable mapping pattern 210. The mapping pattern 210 reflects off tissue in a way that depends on the wavelength of the EMR and the specific anatomical features of the tissue.

The example mapping pattern 210 illustrated in FIG. 10A is a grid array comprising vertical hashing 1002 and horizontal hashing 1004. The example mapping pattern 210 illustrated in FIG. 10B is a dot array. The mapping pattern 210 may include any suitable array for mapping a surface, including, for example, a raster grid of discrete points, an occupancy grid map, a dot array, vertical hashing, horizontal hashing, and so forth. The mapping pattern 210 is emitted by an illumination source 1008, which may originate with an EMR source within the emitter 102 and terminate with an endoscope 110 or tool 108.

As discussed in connection with FIGS. 1A-1C, the distal end of an endoscope 110 may include one or more waveguides 130 that emit EMR that originated at the emitter 102. The mapping pattern 210 may be emitted from these waveguides 130 and projected on to a surface of a tissue. Additionally, one or more tools 108 within a scene may include a waveguide 131 that terminates at a distal end of the tool 108 and/or a side of the tool 108 as shown in FIG. 1A. This waveguide 131 may also emit the mapping pattern 210 on to a surface of a tissue within a scene. In some cases, the tool 108 and the endoscope 110 emit the mapping pattern 210 in concert, and the resultant data frames captured by the image sensor 124 comprise data for tracking the location of the tool 108 within the scene relative to the endoscope 110 or other tools 108.

The emitter 102 may pulse the mapping pattern 210 at any suitable wavelength of EMR, including, for example, ultraviolet light, visible, light, and/or infrared or near infrared light. The surface and/or objects within the environment may be mapped and tracked at very high resolution and with very high accuracy and precision.

The mapping pattern 210 is selected for the desired anatomical measurement scheme, such as three-dimensional topographical mapping, measuring distances and dimensions within a scene, tracking a relative position of a tool 108 within a scene, and so forth. The image sensor 124 detects reflected EMR and outputs a mapping data frame 211 in response to the emitter 102 pulsing the mapping pattern 210. The resultant mapping data frame 211 is provided to a topographical processing 350 algorithm that is trained to calculate one or more of a three-dimensional topographical map of a scene, a distance between two or more objects within the scene, a dimension of an object within the scene, a relative distance between a tool and another object within the scene, and so forth.

The emitter 102 may pulse the mapping pattern 210 at a sufficient speed such that the mapping pattern 210 is not visible to a user. In various implementations, it may be distracting to a user to see the mapping pattern 210 during an endoscopic visualization procedure. In some cases, a rendering of the mapping pattern 210 may be overlaid on a color video stream to provide further context to a user visualizing the scene. The user may further request to view real-time measurements of objects within the scene and real-time proximity alerts when a tool approaches a critical structure such as a blood vessel, nerve fiber, cancer tissue, and so forth. The accuracy of the measurements may be accurate to less than one millimeter.

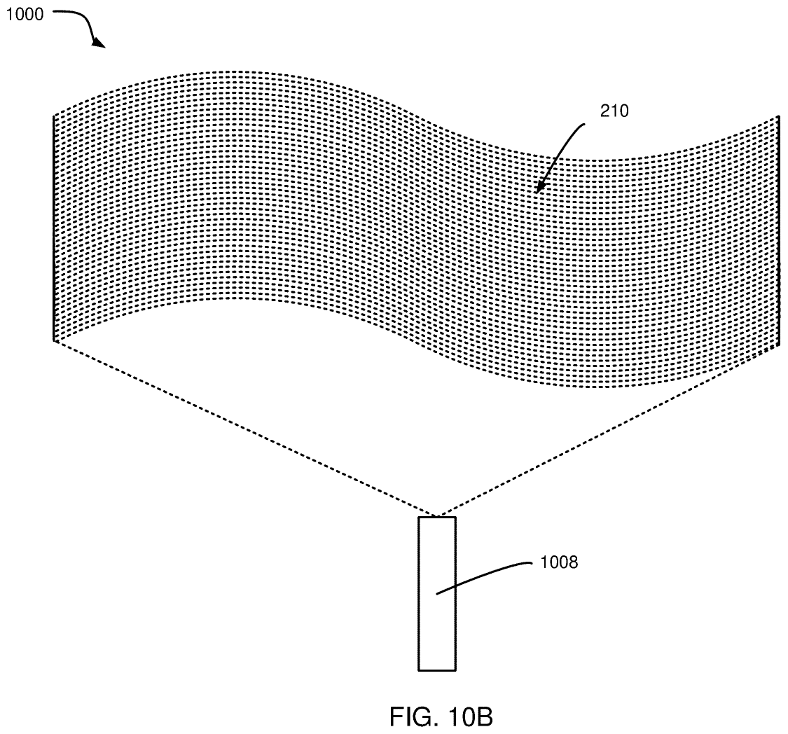

FIG. 11 illustrates a portion of the electromagnetic spectrum 1100 divided into twenty different wavebands. The number of wavebands is illustrative only. In at least one embodiment, the spectrum 1100 may be divided into hundreds of wavebands. The spectrum 1100 may extend from the infrared spectrum 1102, through the visible spectrum 1104, and into the ultraviolet spectrum 1106. Each waveband may be defined by an upper wavelength and a lower wavelength.

Multispectral imaging incudes imaging information from across the electromagnetic spectrum 1100. A multispectral pulse of EMR may include a plurality of sub-pulses spanning one or more portions of the electromagnetic spectrum 1100 or the entirety of the electromagnetic spectrum 1100. A multispectral pulse of EMR may include a single partition of wavelengths of EMR. A resulting multispectral data frame includes information sensed by the pixel array subsequent to a multispectral pulse of EMR. Therefore, a multispectral data frame may include data for any suitable partition of the electromagnetic spectrum 1100 and may include multiple data frames for multiple partitions of the electromagnetic spectrum 1100.

The emitter 102 may include any number of multispectral EMR sources as needed depending on the implementation. In one embodiment, each multispectral EMR source covers a spectrum covering 40 nanometers. For example, one multispectral EMR source may emit EMR within a waveband from 500 nm to 540 nm while another multispectral EMR source may emit EMR within a waveband from 540 nm to 580 nm. In another embodiment, multispectral EMR sources may cover other sizes of wavebands, depending on the types of EMR sources available or the imaging needs. Each multispectral EMR source may cover a different slice of the electromagnetic spectrum 1100 ranging from far infrared, mid infrared, near infrared, visible light, near ultraviolet and/or extreme ultraviolet. In some cases, a plurality of multispectral EMR sources of the same type or wavelength may be included to provide sufficient output power for imaging. The number of multispectral EMR sources needed for a specific waveband may depend on the sensitivity of a pixel array 125 to the waveband and/or the power output capability of EMR sources in that waveband.

The waveband widths and coverage provided by the EMR sources may be selected to provide any desired combination of spectrums. For example, contiguous coverage of a spectrum 1100 using very small waveband widths (e.g., 10 nm or less) may allow for highly selective multispectral and/or fluorescence imaging. The waveband widths allow for selectively emitting the excitation wavelength(s) for one or more particular fluorescent reagents. Additionally, the waveband widths may allow for selectively emitting certain partitions of multispectral EMR for identifying specific structures, chemical processes, tissues, biological processes, and so forth. Because the wavelengths come from EMR sources which can be selectively activated, extreme flexibility for fluorescing one or more specific fluorescent reagents during an examination can be achieved. Additionally, extreme flexibility for identifying one or more objects or processes by way of multispectral imaging can be achieved. Thus, much more fluorescence and/or multispectral information may be achieved in less time and within a single examination which would have required multiple examinations, delays because of the administration of dyes or stains, or the like.

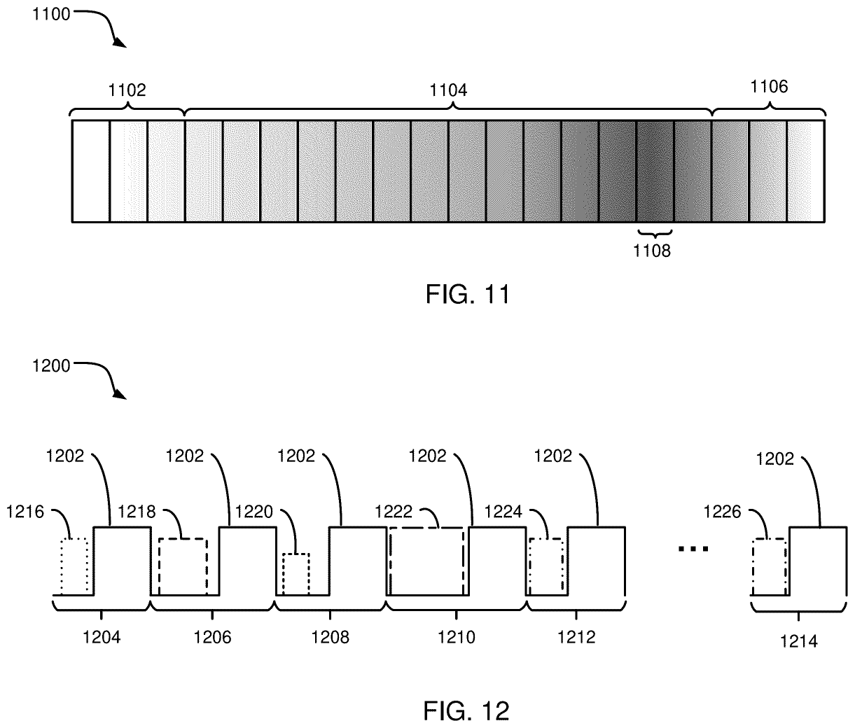

FIG. 12 is a schematic diagram illustrating a timing diagram 1200 for emission and readout for generating an image. The solid line represents readout (peaks 1202) and blanking periods (valleys) for capturing a series of data frames 1204-1214. The series of data frames 1204-1214 may include a repeating series of data frames which may be used for generating mapping, multispectral, and/or fluorescence data that may be overlaid on an RGB video stream. The series of data frames include a first data frame 1204, a second data frame 1206, a third data frame 1208, a fourth data frame 1210, a fifth data frame 1212, and an Nth data frame 1226.

In one embodiment, each data frame is generated based on at least one pulse of EMR. The pulse of EMR is reflected and detected by the pixel array 125 and then read out in a subsequent readout (1202). Thus, each blanking period and readout results in a data frame for a specific waveband of EMR. For example, the first data frame 1204 may be generated based on a waveband of a first one or more pulses 1216, a second data frame 1206 may be generated based on a waveband of a second one or more pulses 1218, a third data frame 1208 may be generated based on a waveband of a third one or more pulses 1220, a fourth data frame 1210 may be generated based on a waveband of a fourth one or more pulses 1222, a fifth data frame 1212 may be generated based on a waveband of a fifth one or more pulses 1224, and an Nth data frame 1226 may be generated based on a waveband of an Nth one or more pulses 1226.

The pulses 1216-1226 may include energy from a single EMR source or from a combination of two or more EMR sources. For example, the waveband included in a single readout period or within the plurality of data frames 1204-1214 may be selected for a desired examination or detection of a specific tissue or condition. According to one embodiment, one or more pulses may include visible spectrum light for generating an RGB or black and white image while one or more additional pulses are emitted to sense a spectral response to a multispectral wavelength of EMR.

The pulses 1216-1226 are emitted according to a variable pulse cycle determined by the controller 104. For example, pulse 1216 may include a white light, pulse 1218 may include a multispectral waveband, pulse 1220 may include a white light, pulse 1222 may include a fluorescence waveband, pulse 1224 may include white light, and so forth.

The plurality of frames 1204-1214 are shown having varying lengths in readout periods and pulses having different lengths or intensities. The blanking period, pulse length or intensity, or the like may be selected based on the sensitivity of a monochromatic sensor to the specific wavelength, the power output capability of the EMR source(s), and/or the carrying capacity of the waveguide.

In one embodiment, dual image sensors may be used to obtain three-dimensional images or video feeds. A three-dimensional examination may allow for improved understanding of a three-dimensional structure of the examined region as well as a mapping of the different tissue or material types within the region.

In an example implementation, a patient is imaged with an endoscopic imaging system to identify quantitative diagnostic information about the patient's tissue pathology. In the example, the patient is suspected or known to suffer from a disease that can be tracked with multispectral imaging to observe the progression of the disease in the patient's tissue. The endoscopic imaging system pulses white light to generate an RGB video stream of the interior of the patient's body. Additionally, the endoscopic imaging system pulses one or more multispectral wavebands of light that permit the system to “see through” some tissues and generate imaging of the tissue affected by the disease. The endoscopic imaging system senses the reflected multispectral EMR to generate multispectral imaging data of the diseased tissue, and thereby identifies the location of the diseased tissue within the patient's body. The endoscopic imaging system may further emit a mapping pulsing scheme for generating a three-dimensional topographical map of the scene and calculating dimensions of objects within the scene. The location of the diseased tissue (as identified by the multispectral imaging data) may be combined with the topographical map and dimensions information that is calculated with the mapping data. Therefore, the precise location, size, dimensions, and topology of the diseased tissue can be identified. This information may be provided to a medical practitioner to aid in excising, imaging, or studying the diseased tissue. Additionally, this information may be provided to a robotic surgical system to enable the surgical system to excise the diseased tissue.

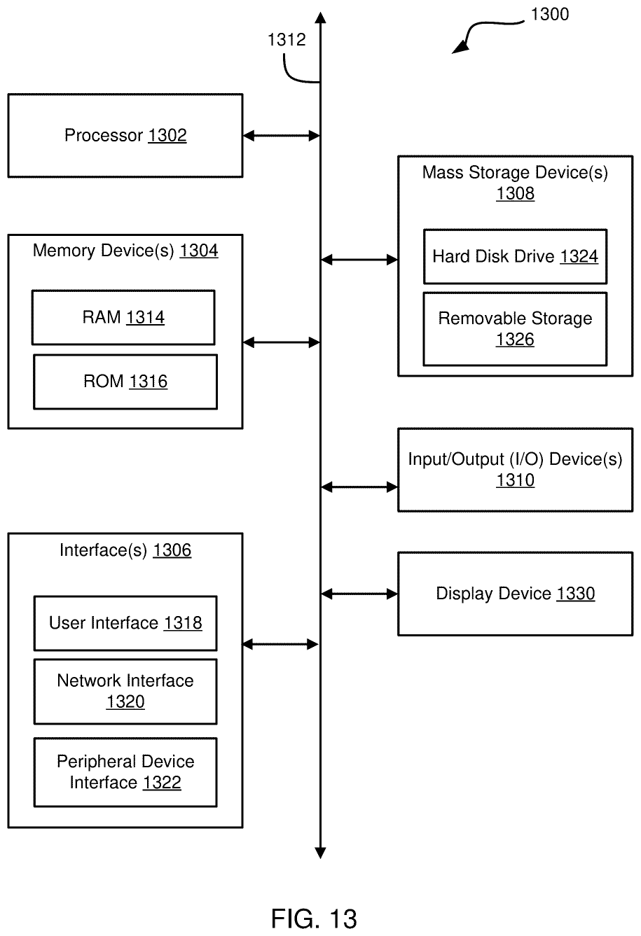

FIG. 13 illustrates a schematic block diagram of an example computing device 1300. The computing device 1300 may be used to perform various procedures, such as those discussed herein. The computing device 1300 can perform various monitoring functions as discussed herein, and can execute one or more application programs, such as the application programs or functionality described herein. The computing device 1300 can be any of a wide variety of computing devices, such as a desktop computer, in-dash computer, vehicle control system, a notebook computer, a server computer, a handheld computer, tablet computer and the like.

The computing device 1300 includes one or more processor(s) 1304, one or more memory device(s) 1304, one or more interface(s) 1306, one or more mass storage device(s) 1308, one or more Input/output (I/O) device(s) 1310, and a display device 1330 all of which are coupled to a bus 1312. Processor(s) 1304 include one or more processors or controllers that execute instructions stored in memory device(s) 1304 and/or mass storage device(s) 1308. Processor(s) 1304 may also include several types of computer-readable media, such as cache memory.

Memory device(s) 1304 include various computer-readable media, such as volatile memory (e.g., random access memory (RAM) 1314) and/or nonvolatile memory (e.g., read-only memory (ROM) 1316). Memory device(s) 1304 may also include rewritable ROM, such as Flash memory.

Mass storage device(s) 1308 include various computer readable media, such as magnetic tapes, magnetic disks, optical disks, solid-state memory (e.g., Flash memory), and so forth. As shown in FIG. 13, a particular mass storage device 1308 is a hard disk drive 1324. Various drives may also be included in mass storage device(s) 1308 to enable reading from and/or writing to the various computer readable media. Mass storage device(s) 1308 include removable media 1326 and/or non-removable media.

I/O device(s) 1310 include various devices that allow data and/or other information to be input to or retrieved from computing device 1300. Example I/O device(s) 1310 include cursor control devices, keyboards, keypads, microphones, monitors or other display devices, speakers, printers, network interface cards, modems, and the like.

Display device 1330 includes any type of device capable of displaying information to one or more users of computing device 1300. Examples of display device 1330 include a monitor, display terminal, video projection device, and the like.

Interface(s) 1306 include various interfaces that allow computing device 1300 to interact with other systems, devices, or computing environments. Example interface(s) 1306 may include any number of different network interfaces 1320, such as interfaces to local area networks (LANs), wide area networks (WANs), wireless networks, and the Internet. Other interface(s) include user interface 1318 and peripheral device interface 1322. The interface(s) 1306 may also include one or more user interface elements 1318. The interface(s) 1306 may also include one or more peripheral interfaces such as interfaces for printers, pointing devices (mice, track pad, or any suitable user interface now known to those of ordinary skill in the field, or later discovered), keyboards, and the like.

Bus 1312 allows processor(s) 1304, memory device(s) 1304, interface(s) 1306, mass storage device(s) 1308, and I/O device(s) 1310 to communicate with one another, as well as other devices or components coupled to bus 1312. Bus 1312 represents one or more of several types of bus structures, such as a system bus, PCI bus, IEEE bus, USB bus, and so forth.

For purposes of illustration, programs and other executable program components are shown herein as discrete blocks, such as block 302 for example, although it is understood that such programs and components may reside at various times in different storage components of computing device 1300 and are executed by processor(s) 1302. Alternatively, the systems and procedures described herein, including programs or other executable program components, can be implemented in hardware, or a combination of hardware, software, and/or firmware. For example, one or more application specific integrated circuits (ASICs) can be programmed to carry out one or more of the systems and procedures described herein.

### EXAMPLES

The following examples pertain to preferred features of further embodiments:

Example 1 is a device for connecting optical and electrical components of an imaging system. The device includes a first fiber optic ferrule dedicated to receiving a first emission of electromagnetic radiation that is emitted by a first source. The device includes a second fiber optic ferrule dedicated to receiving a second emission of electromagnetic radiation that is emitted by a second source. The device includes a data connection component configured to receive bidirectional data communications. The imaging system is such that the first source is tuned to pulse a different waveband of electromagnetic radiation than the second source.

Example 2 is a device as in Example 1, further comprising a multiconductor that comprises: a first plurality of optical fibers in optical communication with the first fiber optic ferrule; a second plurality of optical fibers in optical communication with the second fiber optic ferrule; and a cable in electronic communication with the data connection component, wherein the cable transmits the bidirectional data communications. The device is such that the first plurality of optical fibers is optically independent from the second plurality of optical fibers.

Example 3 is a device as in any of Examples 1-2, wherein the image system comprises a lumen of an endoscope, wherein the first plurality of optical fibers and the second plurality of optical fibers are physically intermixed within one or more of the multiconductor or the lumen of the endoscope.

Example 4 is a device as in any of Examples 1-3, wherein the first plurality of optical fibers and the second plurality of optical fibers are not optically intermixed such that: the first plurality of optical fibers is dedicated to transmitting the first emission of electromagnetic radiation that is emitted by the first source; and the second plurality of optical fibers is dedicated to transmitting the second emission of electromagnetic radiation that is emitted by the second source.

Example 5 is a device as in any of Examples 1-4, wherein the first source emits a plurality of independent pulses of visible electromagnetic radiation.

Example 6 is a device as in any of Examples 1-5, wherein the first source is a white light emitting diode (LED), and wherein the first emission of electromagnetic radiation comprises a plurality of independent pulses of white light.

Example 7 is a device as in any of Examples 1-6, wherein the second source comprises a plurality of narrowband sources that are independently actuatable, and wherein each of the plurality of narrowband sources is tuned to emit a waveband of electromagnetic radiation selected for advanced visualization.

Example 8 is a device as in any of Examples 1-7, wherein the advanced visualization comprises one or more of multispectral visualization or fluorescence visualization, and wherein each of the plurality of narrowband sources is finely tuned to emit electromagnetic radiation within a waveband equal to or narrower than 20 nm wide.

Example 9 is a device as in any of Examples 1-8, wherein the device is an integrated connector and cable for enabling optical and electronic communication between an endoscope and an illumination and visualization controller.

Example 10 is a device as in any of Examples 1-9, wherein the illumination and visualization controller comprises: an emitter comprising the first source, wherein the first source is in optical communication with a first waveguide contact; the emitter further comprising the second source, wherein the second source is in optical communication with a second waveguide contact; and a controller comprising one or more of a field programmable gate array or a computer, wherein the controller is in electronic communication with a data port.

Example 11 is a device as in any of Examples 1-10, wherein the first fiber optic ferrule is configured to interface with the first waveguide contact; wherein the second fiber optic ferrule is configured to interface with the second waveguide contact; and wherein the data connection component is configured to interface with the data port.

Example 12 is a device as in any of Examples 1-11, wherein the device further comprises: a multiconductor comprising a plurality of optical fibers and a cable in electronic communication with the data connection component; a handpiece of an endoscope; and a lumen of the endoscope.

Example 13 is a device as in any of Examples 1-12, wherein the device further comprises a microcontroller unit disposed within the handpiece of the endoscope, wherein the cable and the data connection component enable electronic communication between the microcontroller unit and an external controller.

Example 14 is a device as in any of Examples 1-13, wherein the device further comprises an image sensor disposed within one of the lumen of the endoscope or the handpiece of the endoscope.

Example 15 is a device as in any of Examples 1-14, wherein the lumen of the endoscope comprises two or more concentric channels along a length of a longitudinal axis of the lumen, and wherein the two or more concentric channels comprises: a first innermost channel; and a second channel disposed around the first innermost channel.

Example 16 is a device as in any of Examples 1-15, wherein the image sensor is disposed within the first innermost channel; and wherein the plurality of optical fibers is disposed within the second channel disposed around the first innermost channel.

Example 17 is a device as in any of Examples 1-16, wherein the microcontroller unit is in electronic communication with the image sensor, and wherein the microcontroller unit sets correct registers for the image sensor on a per-frame basis to instruct the image sensor to read out a plurality of data frames according to a sensor cycle.

Example 18 is a device as in any of Examples 1-17, wherein the plurality of optical fibers terminates at a distal end of the lumen of the endoscope.

Example 19 is a device as in any of Examples 1-18, wherein a first portion of the plurality of optical fibers is dedicated to transmitting the first emission of electromagnetic radiation emitted by the first source from the first fiber optic ferrule to the distal end of the lumen of the endoscope; and wherein a second portion of the plurality of optical fibers is dedicated to transmitting the second emission of electromagnetic radiation emitted by the second source from the second fiber optic ferrule to the distal end of the lumen of the endoscope.

Example 20 is a device as in any of Examples 1-19, wherein the first portion and the second portion of the plurality of optical fibers are physically intermixed within the lumen of the endoscope such that each of the first emission of electromagnetic radiation and the second emission of electromagnetic radiation is emitted from the distal end of the lumen of the endoscope with uniform illumination.

It will be appreciated that various features disclosed herein provide significant advantages and advancements in the art. The following claims are exemplary of some of those features.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further, where appropriate, functions described herein can be performed in one or more of: hardware, software, firmware, digital components, or analog components. For example, one or more application specific integrated circuits (ASICs) or field programmable gate arrays (FPGAs) can be programmed to carry out one or more of the systems and procedures described herein. Certain terms are used throughout the following description and claims to refer to particular system components. As one skilled in the art will appreciate, components may be referred to by different names. This document does not intend to distinguish between components that differ in name, but not function.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A device for connecting optical and electrical components of an imaging system, the device comprising:
a first fiber optic ferrule dedicated to receiving a first emission of electromagnetic radiation emitted by a first source;
a second fiber optic ferrule dedicated to receiving a second emission of electromagnetic radiation emitted by a second source; and
a data connection component configured to receive bidirectional data communications;
wherein the first source is tuned to emit a different waveband of electromagnetic radiation than the second source.

2. The device of claim 1, further comprising a multiconductor that comprises:
a first plurality of optical fibers in optical communication with the first fiber optic ferrule;
a second plurality of optical fibers in optical communication with the second fiber optic ferrule; and
a cable in electronic communication with the data connection component, wherein the cable transmits the bidirectional data communications;
wherein the first plurality of optical fibers are optically independent from the second plurality of optical fibers.

3. The device of claim 2, further comprising a lumen of an endoscope, wherein the first plurality of optical fibers and the second plurality of optical fibers are physically intermixed within one or more of the multiconductor or the lumen of the endoscope.

4. The device of claim 3, wherein the first plurality of optical fibers and the second plurality of optical fibers are not optically intermixed such that:
the first plurality of optical fibers is dedicated to transmitting the first emission of electromagnetic radiation that is emitted by the first source; and
the second plurality of optical fibers is dedicated to transmitting the second emission of electromagnetic radiation that is emitted by the second source.

5. The device of claim 1, wherein the first source emits a plurality of independent pulses of visible electromagnetic radiation, and wherein the first source comprises one or more of:
a white light source configured to pulse white light; or
a narrowband source configured to pulse narrowband electromagnetic radiation within a visible wavelength range of the electromagnetic spectrum, wherein the narrowband source is configured to pulse electromagnetic radiation within a waveband comprising a width of 30 nm or less.

6. The device of claim 1, wherein the first source is a white light emitting diode (LED), and wherein the first emission of electromagnetic radiation comprises a plurality of independent pulses of white light.

7. The device of claim 1, wherein the second source comprises a plurality of narrowband sources that are independently actuatable, and wherein each of the plurality of narrowband sources is tuned to emit a waveband of electromagnetic radiation selected for advanced visualization; and
wherein the advanced visualization comprises one or more of multispectral visualization or fluorescence visualization, and wherein each of the plurality of narrowband sources is finely tuned to emit electromagnetic radiation within a waveband equal to or narrower than 20 nm wide.

8. The device of claim 1, wherein the device is a split connector and cable for independently enabling optical and electronic communication between an endoscope and an illumination and visualization controller.

9. The device of claim 1, wherein the device is an integrated connector and cable for enabling optical and electronic communication between an endoscope and an illumination and visualization controller.

10. The device of claim 9, wherein the illumination and visualization controller comprises:
an emitter comprising the first source, wherein the first source is in optical communication with a first waveguide contact;
the emitter further comprising the second source, wherein the second source is in optical communication with a second waveguide contact; and
a controller comprising one or more of a field programmable gate array or a computer, wherein the controller is in electronic communication with a data port.

11. The device of claim 10, wherein the first fiber optic ferrule is configured to interface with the first waveguide contact;
wherein the second fiber optic ferrule is configured to interface with the second waveguide contact; and
wherein the data connection component is configured to interface with the data port.

12. The device of claim 1, wherein the device further comprises:
a multiconductor comprising a plurality of optical fibers and a cable in electronic communication with the data connection component;
a handpiece of an endoscope; and
a lumen of the endoscope.

13. The device of claim 12, wherein the device further comprises a microcontroller unit disposed within the handpiece of the endoscope, wherein the cable and the data connection component enable electronic communication between the microcontroller unit and an external controller.

14. The device of claim 13, wherein the device further comprises an image sensor disposed within one of the lumen of the endoscope or the handpiece of the endoscope; and
wherein the lumen of the endoscope comprises two or more concentric channels along a length of a longitudinal axis of the lumen, and wherein the two or more concentric channels comprises:
a first innermost channel; and
a second channel disposed around the first innermost channel.

15. The device of claim 14, wherein the image sensor is disposed within the first innermost channel; and
wherein the plurality of optical fibers is disposed within the second channel disposed around the first innermost channel.

16. The device of claim 15, wherein the microcontroller unit is in electronic communication with the image sensor, and wherein the microcontroller unit sets correct registers for the image sensor on a per-frame basis to instruct the image sensor to read out a plurality of data frames according to a sensor cycle.

17. The device of claim 12, wherein the plurality of optical fibers terminates at a distal end of the lumen of the endoscope.

18. The device of claim 17, wherein a first portion of the plurality of optical fibers is dedicated to transmitting the first emission of electromagnetic radiation emitted by the first source from the first fiber optic ferrule to the distal end of the lumen of the endoscope; and
wherein a second portion of the plurality of optical fibers is dedicated to transmitting the second emission of electromagnetic radiation emitted by the second source from the second fiber optic ferrule to the distal end of the lumen of the endoscope.

19. The device of claim 18, wherein the first portion and the second portion of the plurality of optical fibers are physically intermixed within the lumen of the endoscope such that each of the first emission of electromagnetic radiation and the second emission of electromagnetic radiation is emitted from the distal end of the lumen of the endoscope with uniform illumination.

20. The device of claim 18, wherein a ratio for a quantity of the first portion and a quantity of the second portion of the plurality of optical fibers is optimized based at least in part on a characteristic of the first source and/or a characteristic of the second source, and wherein the ratio comprises at least one or more of:
about 1:1 the quantity of the first portion to the quantity of the second portion;
about 1.5:1 the quantity of the first portion to the quantity of the second portion;
about 2:1 the quantity of the first portion to the quantity of the second portion;
about 3:1 the quantity of the first portion to the quantity of the second portion; or
about 4:1 the quantity of the first portion to the quantity of the second portion.
